(19) United States
(12) Patent Application Publication
(10) Pub. No.: US 2026/0165750 A1
(43) Pub. Date: Jun. 18, 2026

(54) EXPANDABLE WORM SCREW JACK FOR INSTALLATION BETWEEN UPPER AND LOWER SUCCEEDING ARTICULAR PROCESSES AND HAVING ENHANCED BONE GRIPPING GEOMETRY AND TEETH PROFILES

(71) Applicant: Linares Spinal Devices, LLC, Auburn Hills, MI (US)

(72) Inventors: Miguel A. Linares, Bloomfield Hills, MI (US); Richard W. Easton, Bloomfield Hills, MI (US); Kenneth Williford, Shelby Township, MI (US)

(73) Assignee: Linares Spinal Devices, LLC, Auburn Hills, MI (US)

(21) Appl. No.: 19/533,917

(22) Filed: Feb. 9, 2026

Related U.S. Application Data
(63) Continuation-in-part of application No. 18/131,607, filed on Apr. 6, 2023, now Pat. No. 12,544,234.
(60) Provisional application No. 63/328,309, filed on Apr. 7, 2022.

(51) Int. Cl. A61B 17/70

(52) U.S. Cl. CPC A61B 17/7065; A61B 17/7047; A61B 17/7067

(57) ABSTRACT

A spinal jack adapted for installation between first and second vertebral processes, including a three dimensional and arcuate ergonomic main body constructed from first lower and second upper subset body portions, from which is displaceable an upper body between retracted and expanded positions. Each of the jack halves further includes gripping portions adapted for engaging the vertebral processes and preventing detachment following implantation. A worm gear mechanism is provided for expanding or retracting the jack halves in order to establish a corrected adjusted orientation between the processes.

### CROSS REFERENCE TO RELATED APPLICATIONS

The present application claims the priority of U.S. Ser. No. 18/131,607 filed Apr. 6, 2023. The '607 application claims the priority of U.S. Ser. No. 63/328,309 filed Apr. 7, 2022.

### FIELD OF THE INVENTION

The present invention relates generally to spinal jacks for providing inter-vertebral support. More specifically, the present invention teaches an adjustable spinal jack for installation between superior articular processes of upper and lower succeeding vertebrae. Additional variants include reconfiguring the implant body for other non-vertebral applications, such as in use with first and second segmented bones associated with any of a humerus, femur or the like.

### BACKGROUND OF THE INVENTION

Spinal jacks designs are known in the prior art for providing adjusted and secure positioning support between succeeding spinal vertebra. Examples of these are depicted in each of Linares U.S. Pat. No. 8,623,056 and Linares U.S. Pat. No. 8,585,738.

Other examples from the prior art include the inter-vertebral body fusion device of Jimenez et al., US 2011/0160861 which incorporates a drive mechanism configured to interface with coaxial screw gear sleeve mechanisms, for causing the device to distract.

Woodworth, US 2016/0135851 teaches an interlaminar, interspinous stabilization device for placement between the spinous processes of adjacent cervical vertebrae and optionally secured to the lamina using bone screws or crimped or rigidly fixed to the spinous process.

### SUMMARY OF THE PRESENT INVENTION

The present invention discloses a spinal jack adapted for installation between first and second vertebral processes including a body constructed from upper and lower inter-displaceable body portions, from which is displaceable an upper body between retracted and expanded positions. Without limitation, the lower body portion (also termed a main body) can include first and second subset portions which, upon final assembly, are laser welded along its joining interface.

Each of the jack halves further includes gripping portions adapted for engaging the vertebral processes and preventing detachment following implantation. The gripping portions each further include spaced apart sides and an interconnected recessed end collectively defining a pocket adapted to receive the vertebral process therebetween.

A worm gear mechanism is provided for expanding or retracting the jack halves of the installed jack, this in order to establish a corrected adjusted orientation between the processes. The worm gear mechanism further includes a central horizontally arrayed and rotatable worm having a bit engaging portion integrally being formed with the worm and projecting exterliorly from a surface of the main body.

The central rotatable worm is configured as a centrally disposed screw gear with a tool bit engageable hex protrusion, with rotation of the central worn interengaging with a pair of outer crosswise disposed worm gears, the worm exhibiting an outer array of gear teeth meshing in bevel fashion with the crosswise arranged exterior gear teeth configured upon each of a pair of outer crosswise disposed worm gears arranged upon opposite sides of the central worm. Lift screws can be rotatably and threadably engaged with opposing inner threads of the outer gears and include extending stem portions which secure the upper body and which, upon actuation of the central worm, result in outward displacement of the upper body portion relative to the lower body portion. Without limitation, the terms “worm” and “worm gear” in the context of the overall mechanism can be utilized interchangeably, with the objective being to prevent inadvertent displacement of the jack halves through unintended rotation of the gear mechanism, and once the desired orientation has been achieved.

In certain variants, the outer crosswise disposed bevel gears and inner lift screws/stem portions can be integrated into redesigned lift screws, with the inner circumferential threads reconfigured directly into each of the upper and lower body portions, and in order to simultaneously displace the upper and lower body portions upon actuation of the central worm, and as opposed displacing only the upper body portion relative to the lower body portion. The effect of simultaneous displacement is to reduce by half the number of turns of the central worm gear in effectuating a length displacement between the upper and lower body portions.

Other features include repositioning the jack halves in a depth-offsetting fashion in order to inwardly redirect inwardly compression forces exerted by the vertebral processes. The gripping portions further include gripping teeth distributed along the opposing gripping surfaces and facilitating unidirectional installation of the processes along with preventing detachment of uneven surfaces of the processes once installed. A further advantage of the worm gear arrangement is that it prevents reverse/collapsing adjustment in response to inward forces applied by the superior articular processes on the respective jack halves.

The body and inter-expandable jack halves further can be constructed of any material and typically a medical grade Titanium which is bio-compatible and FDA approved for surgical implants. The lower body portion incorporates a recessed cavity for receiving the central worm gear and outer bevel gears. Also provided are additional cavities configured into the body portions for seating the extending stems.

Additional aspects of the invention include the ability to 3D print the body components, including each of the upper and lower body portions. Without limitation, the additive printed material can again include Titanium in addition to other suitable medical grade material including other metals or polymeric composites.

A further advantage of additive printing of the body components is the ability to modify the density of the material, this including reducing a surface material distribution through such as a “latticing” technique which envisions gaps in the printing of the surface layers, such as which can include the gripping portions and spinal process receiving pockets. In this fashion, in-growth of bone into the latticed areas is promoted which enhances the engagement of the implant.

The additive printing techniques employed for producing the implant body components permit the configuration of the teethed gripping portions according to varying sizes and directions, such as increasing in size in both inward and/or downward seating directions in order to enhance the initial seating engagement of the spinal processes into the implant gripping pockets. Other modifications in the gripping teeth include individual rows being provided with incrementing height, with the shortest being the initial row engaging the vertebral bone, each succeeding row of teeth subsequently engaging and digging a little deeper into the bone as a fresh grip and so that, upon completed installation, all of the individual rows of teeth are biting into new bone during installation of the spinal jack into the succeeding vertebral processes.

Other features include providing variations in the design of the multiple rows of gripping teeth configured in opposing plural arrangement within the upper and lower “U” shaped receiving pockets defined in the upper and lower subset riser bodies. This includes the teeth being directional, meaning they will easily push in, but catch or grab in response to a reverse outward force.

Additional to deformation engagement of the seated implant pockets around the processes, such as through the use of crimping pliers, other fastener options for securing the upper and lower implant bodies to the vertebral processes include the use of tubular shaped rivets, which can be installed following initial drilling through the bone aligning with the apertures in the seating pockets. The rivets can include surface apertures for facilitating bone in-growth and can also be hollowed, in the latter instance, permitting the use of retention cables for providing additional retention. Removal of previously installed rivets can be further accomplished through the use of a width directed tool bit which is seats within and laterally displaces the rivet.

The fixation pins or rivets can also be redesigned to be inserted between spaced apart and aligning apertures configured into upper and lower pairs of ears which are configured into the upper and lower body portions. An associated fixation tool provides for forcibly installing the rivet through the underlying vertebral processes, without the requirement of pre-drilling. The rivet includes bone growth through-holes or apertures around its circumference for facilitating in-growth of bone in order to permanently secure the pins in place.

Without limitation, the pins/rivets can be modified to exhibit a roll or split pin design with the center diameter of the roll pin being slightly larger than the outer edges, such that, when pressing through the spaced apart apertures and the bone, it will expand into position and be restrained from sliding out from between the apertures. The circular extending opposing edges of the rivet exhibit a very thin wall thickness so as to establish a knife edge which can be pushed through the bone, thereby eliminating the need for any pre-drilling through the bone.

Also disclosed is an installation tool that provides for locating and resistive seating of the spinal implant against the spinal processes of the successive vertebrae, such as via the rows of gripping teeth and prior to final installation utilizing the rivets and fixation tool. A forward end of the installation tool exhibits a rectangular shape compressed between the upper and lower implant body portions. A bit engaging socket is located within the forward end open interior and seats over the hex bit portion for controlling the displacement of the spinal jack halves.

Upon initial linear seating of the implant pockets to the spinal processes, such including exerting forward impact forces upon the tool for resistively seating the gripping pockets to the spinal processes, a rear handle end of the tool is removed to reveal a rear projecting end of a tool bit driver extending within the tool interior to the forward located bit engaging socket, such that the rotation of the rear driver rotates the hex bit to initiate separation between the upper and lower implant bodies.

Additional variants of the spinal jack riser body designs include modification to each of the worm, worm gear and lift screw mechanics, this being reliant upon the application and associated mechanical restraints, and in order to provide multiple ways of lifting/separating the upper and lower halves with respect to one another in order to establish a desired separation distance between the successive vertebrae.

Versions of the inter-vertebral spinal jack also include the upper and lower body portions or halves being symmetrically constructed to permit being implanted in either of first upright or second rotated (upside-down) positions. The symmetrical design also makes bone preparation easier before device insertion. The upper and lower vertebrae can be prepped the same and assembled without the use of screws in favor of laser welding or fusing of the different titanium parts, with no additional weld material added.

An associated set of preparation, implantation and fixation tools can be provided for successively prepping the installation area for the implant, installing the implant and then fixing it in place with the rivet affixation tool. This can include the set of tools being provided for potentially preparing and installing a variety of different jack implants.

In a basic construction, the preparation tool includes a pair of manually or electric motor driven rotary blades which operate to condition the surfaces of the affected vertebrae for seating respective upper and lower spinal body halves. As noted, the configuration of the rotary blades can be such that they can be utilized to symmetrically shave both the upper and lower vertebrae for receiving each of symmetrically constructed upper and lower spinal jack halves.

The implantation tool succeeds the preparation tool for initially locating and placing the spinal jack halves to the previously preparatory conditioned surfaces of the vertebra, following which the fixation tool is employed for driving the rivets between the upper and lower ears or lobes of the spinal body halves and through the intervening bone, again without the need for pre-drilling holes between the ear lobe recesses.

The present invention also contemplates reconfiguring the implant body for other non-vertebral applications, such as in use with first and second segmented bones associated with any of a humerus, femur or the like. In this instance, the U-shaped vertebral process seating pockets are reconfigured as inner elongated bodies which seat within the marrow interiors of the elongated bone segments for bonding the upper and lower body portions to the opposing end faces of the bone segments.

### BRIEF DESCRIPTION OF THE DRAWINGS

Reference will now be made to the attached drawings, when read in combination with the following detailed description, wherein like reference numerals refer to like parts throughout the several views, and in which:

FIG. 1 is a perspective view of a spinal worm screw jack according to one non-limiting variant and having upper and lower body portions shown in a retracted position prior to being installed between succeeding superior articular processes associated with upper and lower consecutive spinal vertebra;

FIG. 2 is a similar perspective view to that shown in FIG. 1 of a variant of spinal worm screw jack exhibiting increased distribution of unidirectional gripping teeth exhibited upon upper and lower “U” shaped channel defining gripping surfaces;

FIG. 3 is a cutaway taken along line 3-3 of FIG. 1 and depicting the interior worm screw and gearing for separating the upper and lower bodies and including the input worm screw, outer slaved worm gears and coaxially inter-rotating and axially displacing screws provide for fine incremental adjustment of the respective half halves;

FIG. 4 depicts the spinal worm screw jack of FIG. 1 in an outwardly expanded position;

FIG. 5 is a cutaway view taken along line 5-5 of FIG. 4 of the screw jack bodies and worm screw gearing in the expanded position;

FIG. 6 is an illustration similar to FIG. 1 and showing the tabs associated with the upper and lower jack body gripping portions receiving inwardly directed screws or push in clips for anchoring to the articular processes;

FIG. 7 is an illustration similar to FIG. 4 and depicting an intermediate cutaway region associated with the lower body for revealing the input hex input worm screw, outer slaved worm gears and coaxially inter-rotating and axially displacing screws provide for fine incremental adjustment of the respective half halves;

FIG. 8 is an environmental view depicting the spinal jack installed between a pair of succeeding spinal vertebrae according to one non-limiting application of the present invention;

FIG. 9 a perspective view of a spinal worm screw jack according to a further non-limiting variant depicting the first and second body portions shown in a retracted position, as well as depicting a tubular shaped mounting rivet for securing through aligning apertures formed in spaced apart and gripping pocket defining tabs associated with an upper selected of the spinal jack body portions, the rivet exhibiting in-growth promoting apertures when engaged through the spinal vertebrae processes depicted in FIG. 8;

FIG. 10 is an overhead and partially cutaway depiction of the spinal jack of FIG. 9 showing a width directed rivet removal tool bit prior to seating and laterally displacing the rivet from between the tabs and the gripped spinous process;

FIG. 11 presents a succeeding view to FIG. 10 and depicting in perspective the removal of the tubular shaped rivet;

FIG. 12 is a similar view to FIG. 10 and depicting the rivet in a substantially removed position in which the width directed tool bit is substantially translated between the gripping tabs;

FIG. 13 is an illustration similar to FIG. 9 and depicting the tubular mounting rivet in a pre-installation position, along with an alternately configured width directed rivet installation tool bit, the rivet depicting engagement embossments configured upon the exterior circumference of the tubular rivet in order to define a correct lateral mounting position shown in FIG. 9;

FIG. 14 is an overhead view similar to FIGS. 10 and 12 and, progressing from FIG. 13, depicting the pre-installed tubular rivet seated over the width directed installation tool bit prior to insertion;

FIG. 15 is a substantial repeat of FIG. 9 showing the tubular shaped mounting rivet engaged and prior to retraction of the width directed installation tool;

FIG. 16 is an overhead and partially cutaway view of FIG. 15, similar in presentation to each of FIGS. 10, 12, and 14, and again showing the rivet fastener installed through the pre-drilled passageway formed in the vertebral process;

FIG. 17 is an environmental illustration similar to that shown in FIG. 8 and depicting a crimping option for deforming and affixing the extending tabs defining each of the upper and lower gripping pockets against the spinous processes, such as additionally or alternative to the use of mounting fasteners of FIG. 9;

FIG. 18 provides a perspective of an installation tool which provides for locating and resistive seating of the spinal implant against the spinal processes of the successive vertebrae, such that, and upon initial linear seating of the implant pockets to the spinal processes, exerted impact forces upon the tool resistively seating the gripping pockets against the spinal processes, whereupon a rear handle end of the tool is removed to reveal a rear projecting end of a tool bit driver extending within the tool interior to the forward located bit engaging socket, with successive rotation of the rear driver rotating the hex bit to initiate separation of the upper and lower implant bodies;

FIG. 19 is a succeeding view to FIG. 18 and depicting a removable rear handle end of the tool revealing a rear projecting end of a tool bit driver extending within the tool interior to a forward located bit engaging socket;

FIG. 20 is a further succeeding view to FIG. 19 and depicting a rotation of the rear driver in order to rotate the hex bit to initiate separation of the upper and lower implant bodies;

FIG. 21 is an enlarged view of forward area 21 of the implant tool depicted in FIG. 20 and depicting the rotating expansion of the upper and lower spinal body portions;

FIG. 22 depicts a pair of implanted spinal jacks, such as individually shown in FIG. 9, with the further installation of retention cables extending through the hollowed tubular rivets for providing additional retention;

FIG. 23 is a vertical cutaway of a selected implanted spinal implant or jack such as shown in FIG. 22 and exhibiting a solid tubular mounting rivet;

FIG. 24 is a similar view to FIG. 23 and depicting use of a tubular mounting rivet;

FIG. 25 is a further succeeding view to FIGS. 23-24 and depicting hollowed tubular mounting rivet with bone in-growth apertures such as depicted in FIG. 9 et seq.;

FIG. 26 is a yet further succeeding view depicting a modified tubular rivet with solid end portions and partially open middle;

FIG. 27 is a rear plan view of a selected spinal implant jack and depicting an arrangement of gripping teeth associated with the process engaging pockets configured in each of the upper and lower body portions;

FIG. 28 is a horizontal cutaway view taken along line 28-28 of FIG. 27 and better illustrating the configuration of the base surface gripping teeth associated with the upper process engaging pocket;

FIG. 29 presents a further perspective and vertical cutaway of the spinal implant jack according to the present invention in a fully closed position;

FIG. 30 is a succeeding view to FIG. 29 depicting the upper and lower spinal implant body portions in a substantially expanded configuration;

FIG. 31 presents a depiction of a spinal implant jack exhibiting aperture patterns distributed across the gripping pockets, such as which can be accomplished as part of a three dimensional or additive printing process for forming each of the individual outer body components, including each of the first and second subset lower body portions and displaceable upper body, without limitation the additive printed material including a titanium or other suitable medical grade material including other metals or polymeric composites;

FIG. 32 illustrates an expanded area referenced in FIG. 31 of the spinal process gripping pocket associated with the upper body portion and better depicting the latticing of the surface layers in order to promote bone in-growth following implantation, the additive printing techniques employed for producing the implant body components permit the configuration of the teethed gripping portions according to varying sizes and directions, such as increasing in size in both inward and/or downward seating directions in order to enhance the initial seating engagement of the spinal processes into the implant gripping pockets;

FIG. 33 presents a perspective environmental of a further version of an implant for non-vertebral applications, such as in use with first and second segmented bones associated with any of a humerus, femur or the like;

FIG. 34 presents a succeeding illustration to FIG. 33 in which the upper and lower implant bodies are shown in an expanded configuration;

FIG. 35 is a vertical cutaway taken along line 35-35 of FIG. 33 and depicting the configuration of the implant in which the U-shaped vertebral process seating pockets of the earlier embodiments are reconfigured as inner elongated bodies which seat within the marrow interiors of the elongated bone segments, for bonding the upper and lower body portions to the opposing end faces of the bone segments;

FIG. 36 is a vertical cutaway taken along line 36-36 of FIG. 34 and depicting the expanded configuration established between the implant upper and lower body portions for establishing a desired and adjustable separation distance between the bone segments;

FIG. 37 presents a perspective view of a spinal worm screw jack similar to that previously shown in FIG. 27 and according to a further non-limiting variant and having upper and lower body portions shown in a retracted position prior to being installed between succeeding superior articular processes associated with upper and lower consecutive spinal vertebra, along with a split rivet for securing between aligning apertures formed in spaced apart and gripping pocket defining tabs associated with the upper and lower spinal jack bodies, the rivets exhibiting in-growth promoting apertures when engaged through the spinal vertebrae process;

FIG. 38 presents a further perspective and vertical cutaway of the spinal implant jack of FIG. 37, similar to that previously shown in FIG. 29, according to the present invention in a fully closed position;

FIG. 39 presents a similar view to FIG. 38 and showing the spinal implant jack in an expanded position similar to FIG. 30;

FIG. 40 presents a perspective view of a further variant of a spinal worm screw jack and again having upper and lower body portions shown in a retracted position prior to being installed between succeeding superior articular processes associated with upper and lower consecutive spinal vertebra, along with a split rivet for securing between aligning apertures formed in spaced apart and gripping pocket defining tabs associated with the upper and lower spinal jack bodies, the rivets exhibiting in-growth promoting apertures when engaged through the spinal vertebrae process;

FIG. 41 is a perspective view of a further variant of a spinal worm screw jack having upper and lower body portions further including bone gripping surfaces adapted for engaging the vertebral processes, the bone gripping surfaces each further including a “U” shaped pocket, aligning apertures formed through spaced extending tabs defining each of the “U” shaped pockets, the upper and lower body portions having a shape and configuration which permits installation in either of first and second one-hundred and eighty degree rotated positions for securing to the first and second vertebral processes;

FIG. 42 is a cutaway view taken along line 42-42 of FIG. 41 and showing the spinal implant jack in a closed position with a further variant of worm gears and lift screws for elevating the upper spinal body portion relative to the lower spinal body portion;

FIG. 43 presents an expanded position of the spinal implant of FIG. 42;

FIG. 44 is a perspective view of a further variant of a spinal worm screw jack, similar in respects to that previously shown in FIG. 41, and again having upper and lower body portions further including bone gripping surfaces adapted for engaging the vertebral processes, the bone gripping surfaces each further including a “U” shaped pocket, aligning apertures formed through spaced extending tabs defining each of the “U” shaped pockets, the upper and lower body portions having a shape and configuration which permits installation in either of first and second one-hundred and eighty degree rotated positions for securing to the first and second vertebral processes;

FIG. 45 is a cutaway view taken along line 45-45 of FIG. 44 and showing the spinal implant jack in a closed position with a further variant of worm gears and lift screws for elevating the upper spinal body portion relative to the lower spinal body portion;

FIG. 46 presents an expanded position of the spinal implant of FIG. 45;

FIG. 47 presents a sectional view of a non-limiting example of a worm gear arrangement according to the present invention, such as previously depicted in the spinal jack of FIGS. 45-46;

FIG. 48 presents a sectional view of a further non-limiting example of a worm gear arrangement in which individual pairs of upper/lower split stems are contained within the outer rotatable gears for simultaneously displacing the upper and lower spinal body portions;

FIG. 49 presents a sectional view of a further non-limiting example of a worm gear arrangement in which individual split stems are contained within the outer rotatable gears for displacing the upper body relative to the lower spinal body;

FIG. 50 presents a cutaway taken along line 50-50 of FIG. 48 and illustrating the internal mating rotary threaded arrangement established between the opposing inner ends of each of the individual pairs of upper/lower split stems contained within the outer rotatable gears, shown in the closed position and again for simultaneously displacing both the upper and lower bodies upon rotation of the central worm gear;

FIG. 51 presents a cutaway view taken along line 51-51 of FIG. 49 of the further non-limiting example of worm gear arrangement and illustrating the internal mating rotary threaded arrangement established between the individual split stems contained within the outer rotatable gears provides for displacing the upper spinal body portion relative to the lower spinal body portion, again upon rotation of the central worm gear;

FIG. 52 presents an environmental illustration of a bone preparation tool forming a part of a tool kit assembly including each of preparation, implantation and fixation tools for installing a spinal implant jack according to the present invention and depicting the preparation tool simultaneously grinding the opposite surfaces of the pair of successive spinous processes in order to establish an installation area prior to installation of the spinal jack;

FIG. 53 depicts a rotated view of the bone preparation tool and depicting the pair of electric motor driven rotary blades which operate to condition the surfaces of the affected vertebrae process for seating respective upper and lower spinal body halves, the configuration of the rotary blades being utilized to symmetrically shave both the upper and lower vertebrae for receiving each of reversible spinal jacks such as previously depicted in FIGS. 41 and 44;

FIG. 54 presents a perspective view of the split rivet according to the present invention as initially shown in FIG. 37 and depicting a split tubular shape along its extending length and including apertures distributed across a width and circumference thereof for facilitating bone in-growth;

FIG. 55 is a rotated plan view of the split rivet of FIG. 54 and showing a larger central diameter in comparison to first and second end located diameters for affixing in position within the vertebral bone.

FIG. 56 presents an initial implantation step of a spinal jack by an implantation tool similar in respects to as previously shown in FIG. 18 and illustrated environmentally in which the user manipulates the tool by twisting and turning, with the spinal jack clamped at the forward end as previously depicted in FIG. 21, and in order to seat the upper and lower spinal jack portions upon the bone prepared surfaces of the upper and lower spinal processes, this further the redesigned handle being removed in order to permit the user to pound an exposed and flattened circular end of the tool with such as a mallet in order to position the spinal jack halves as close to the base of the spinous processes as is possible;

FIG. 57 is succeeding view of FIG. 56 and depicting the handle reattached to the implantation tool of FIG. 56;

FIG. 58 further depicts the fixation tool employed for driving the rivets between the upper and lower ears or lobes of the spinal body halves and through the intervening bone of the spinous processes, again without the need for pre-drilling holes through the bone between the ear lobe recesses; and

FIG. 59 depicts a final installation step using the implantation tool, and following either of the installation steps of FIG. 57 or FIG. 58, and by which the handle is rotated in order to expand the upper and lower spinal bodies into a desired expanded position for properly aligning the vertebrae using the spinal jack.

### DETAILED DESCRIPTION OF THE PREFERRED EMBODIMENTS

With reference to the attached illustrations, the present invention discloses a variety of implant devices utilizing an expandable worm gear mechanism for providing incremental and secure adjustability in order to define a desired spatial separation distance between first and second bones. As will be described, and in a first application, an adjustable spinal jack is provided for installation between superior articular processes of upper and lower succeeding vertebrae. In a further non-limiting application, the implant can be redesigned to define a spatial separation distance between first and second bone segments, such as associated with a femur, humerus or the like. In any application, the present invention provides an expandable spinal jack which overcomes many of the disadvantages of the prior art and provides an effective solution for stabilizing a given orientation established between the first and second bone, processes or bone segments.

As will be further described, the spinal jack designs described herein further permit adjustment, at any future time following initial surgical implantation, in a minimally invasive fashion and in order to re-adjust the spatial positioning established between the upper and lower separable halves or sections, such as in order to compensate and correct for future/downstream vertebral complications following the initial implantation of the spinal jack.

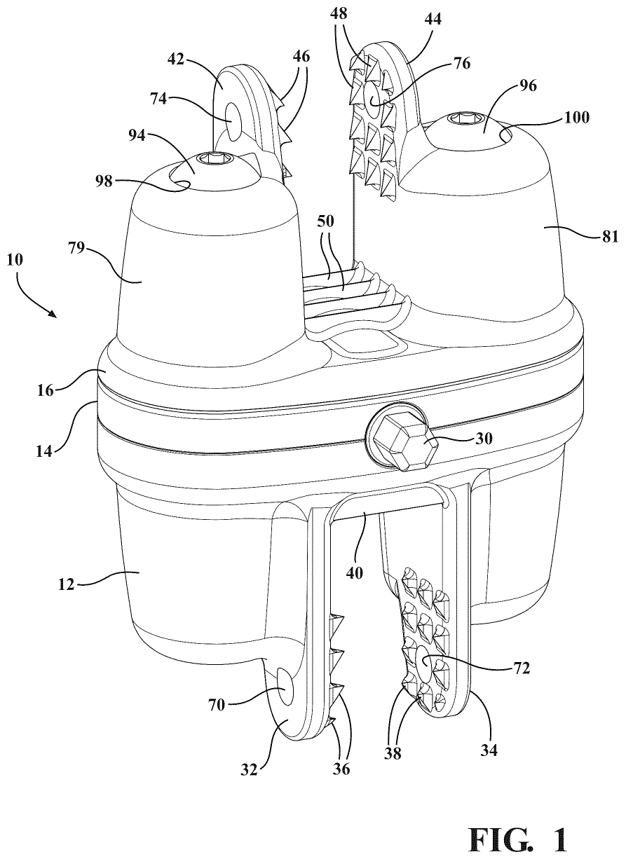

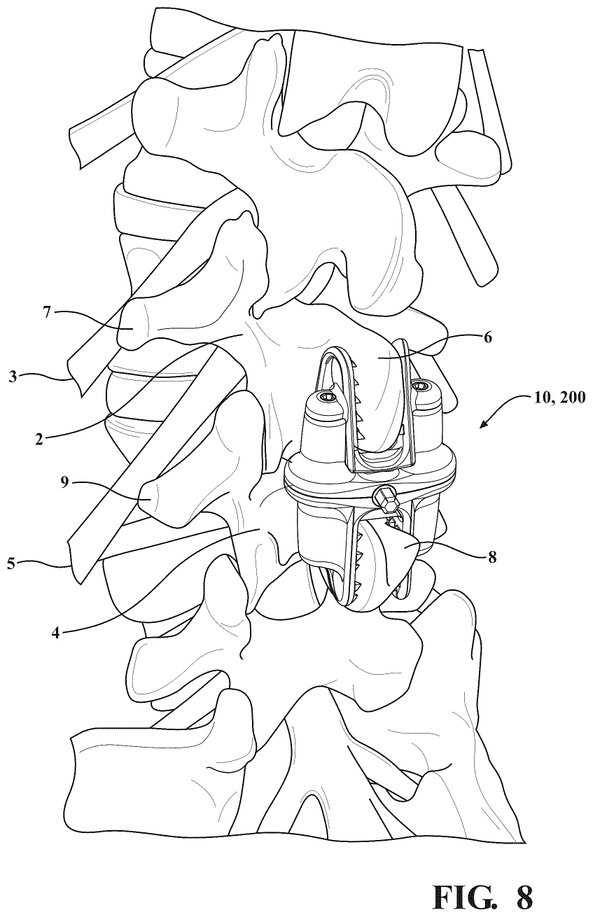

Proceeding to FIG. 1, an illustration is generally shown of a jack implant 10 shown in a retracted position according to a first spinal implant configuration. As depicted in FIG. 8, an environmental view is shown depicting the spinal jack (such as at 10 or 200 according to alternating embodiments) installed between a pair of succeeding spinal vertebrae 2 and 4 according to one non-limiting application of the present invention. As shown, the vertebrae 2 and 4 each include an arrangement of processes, including spinous processes 6 and 8 to which the implant can be mounted. Without limitation, the implants can also be reconfigured to mount to succeeding transverse processes (see at 7 and 9 respectively) associated with the vertebrae 2 and 4.

As is known, vertebrae are bones located within the vertebral column which, in humans, encompass a series of thirty three bones that run from the base of the skull to the coccyx (not shown). As is further known, the irregularly shaped bones form the roughly S-shape of the spinal cord. Between each vertebra is an intervertebral disc, which helps provide shock absorption and protect the vertebrae.

At the base of the skull, the vertebral column starts with the cervical vertebrae. There are seven of these, numbered C1 through C7, which allow the neck the full range of motion they have. The thoracic vertebrae are the next vertebrae, larger than the cervical vertebrae, and moving down the spinal column, these articulating with the ribs, helping to protect the chest cavity containing the heart and lungs. The next five vertebrae are the lumbar vertebrae, the largest and greats weight supporting of the vertebrae, producing a natural curvature to the spine. Lumber vertebrae further allow for flexion, extension, and side-bending.

The remaining vertebrae are the five vertebrae that form the fused sacrum, as well as the three to five vertebrae that form the coccyx or tailbone. The sacral and coccygeal vertebrae do not have intervertebral discs. These bones are sometimes referred to as the caudal vertebrae and have the most variation in number, with some species having a few and others having numerous caudal vertebrae.

As is also depicted in FIG. 8, the vertebrae provide attachment points for muscles and ligaments, allowing many of the motions that the body is able to go through, such as bending and twisting. The vertebrae also protect the spinal cord, which runs down openings in the vertebrae (branches of the spinal cord being shown at 3, 5 et seq.). As a result of this protection, the risk of damage due to trauma and everyday activities is minimized. Also, openings known as foramina are provided which allow the spinal nerves to pass through, providing nervous innervation to different tissues.

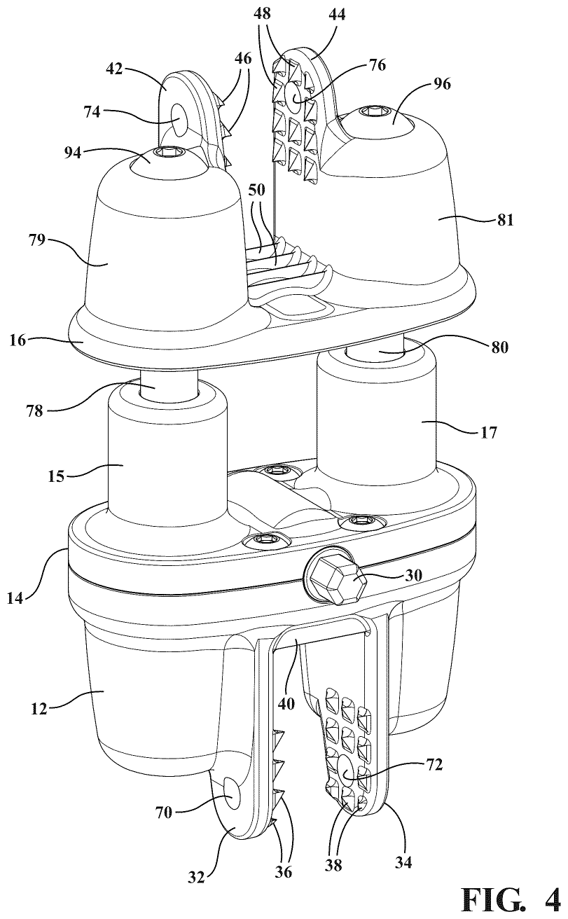

In combination with the expanded positions of FIGS. 4, 5 and 7, the spinal jack can be provided in a set of varied sizes for implantation into each of the cervical, thoracic and lumbar sections. In each instance, the implant or jack 10 includes a three dimensional and arcuate ergonomic lower body portion constructed from first 12 and second 14 subset body portions, from which is displaceable an upper body portion 16 (as best shown in FIG. 4). The subset body portion 14 further includes superstructure portions 15 and 17. The body portions, as will be further described, can be machined, injection molded or additive printed. Interior components of the worm gear mechanism, supporting and displacement stems and the like can also be provided as any machined or stamped construction not limited to material composition however typically including a medical grade sanitary material not limited to titanium.

The subset lower body portions 12/14 and upper displace-able body portions 16 are each constructed of a suitable sanitary medical grade material not limited to any of a metal (e.g. typically titanium) or other plastic composition. As will be further described in subsequent variants, the upper body portion 16 and subset lower body portions 12/14 can be individually three dimensionally (3D) or additive printed, permitting greater detail and material variation (e.g. surface latticing as will be further described) than which is capable with other existing forming (e.g. stamping and molding) techniques.

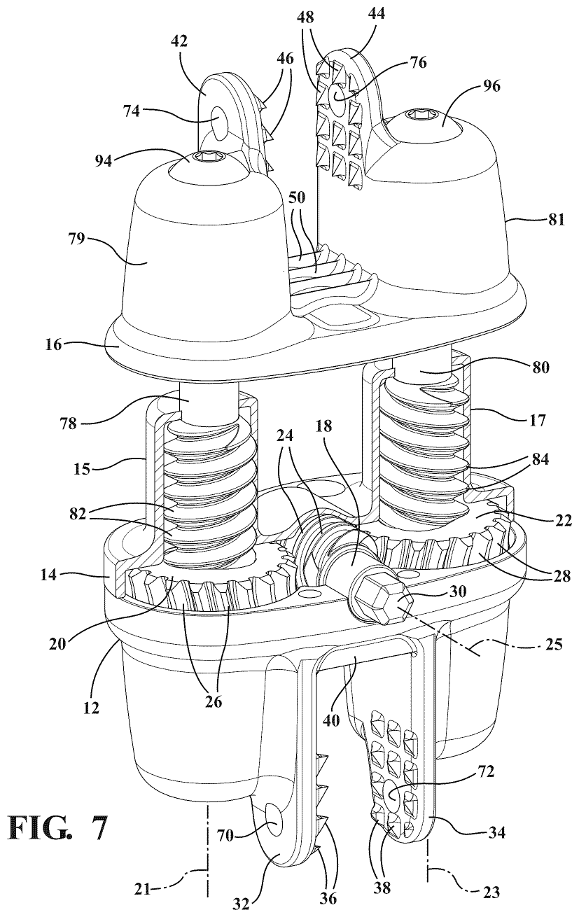

As best shown in the cutaway view of FIG. 7, the subset assembled lower body portions 12/14 (which can be sonic welded or otherwise secured) define a package interior space for collectively seating a worm gear mechanism including each of a central worm 18 and inter-engaging and rotationally actuated outer located worm gears 20 and 22. The outer gears 20/22 are arranged so that a rotational centerline axis of each (see at 21 and 23 in FIG. 7) is arranged in a perpendicular direction relative to a rotational centerline (further at 25) of the central worm 18.

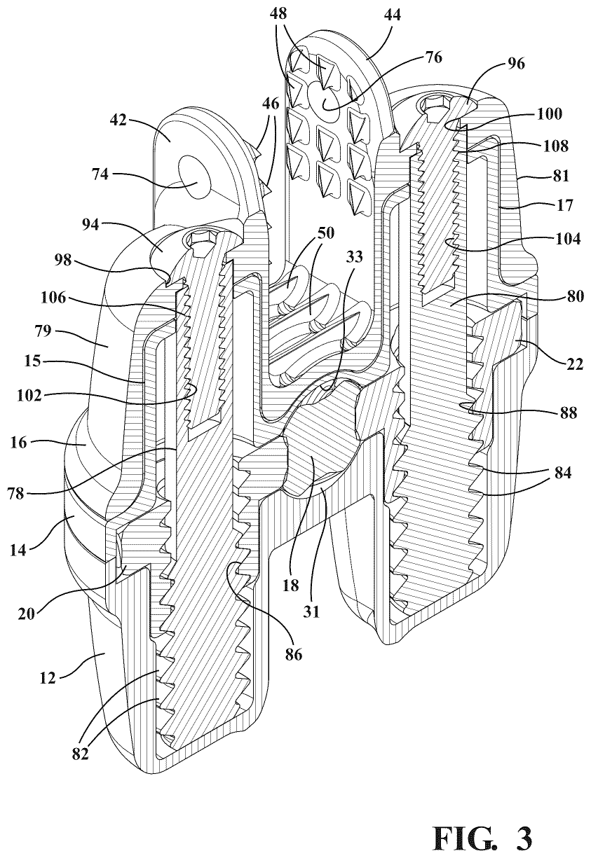

As shown in FIGS. 4 and 7, the central worm 18 includes a spiral array of gear teeth 24 extending along its generally horizontal length which mesh with the crosswise arranged and annular arrayed gear teeth, shown at 26 arranged upon outer gear 20 located on one side of the worm gear 18, and further at 28 arranged upon the other outer gear 22 arranged on the opposite side of the worm 18. A projecting hex bit portion 30 is integrally formed with an axial end of the central worm 18 and which seats within and (optionally) projects forwardly from aligning annular rim portions (see at 31 and 33 in FIG. 3 cutaway) defined between the opposing end faces of the subset body portions 12/14.

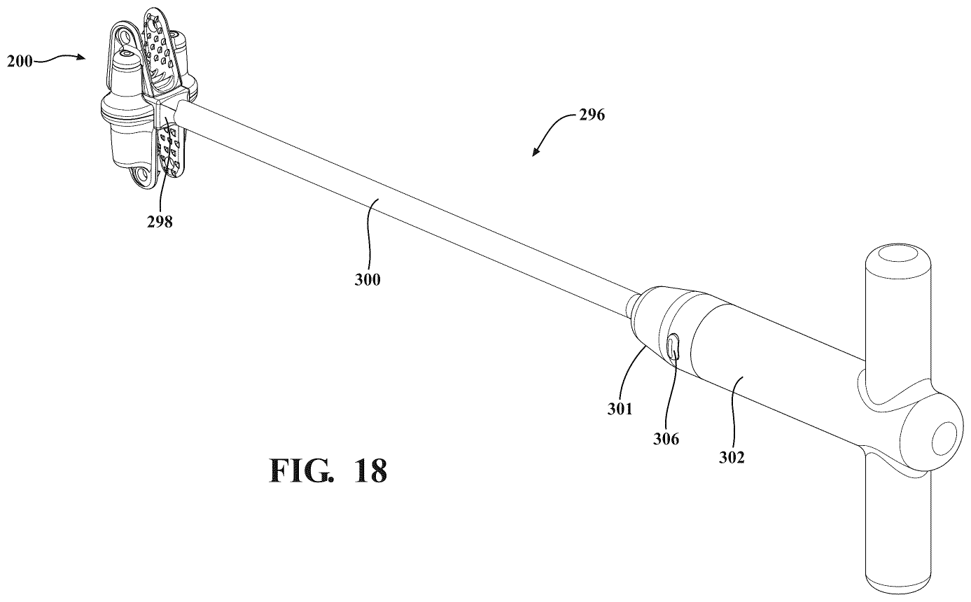

In this fashion, a socket style tool bit (reference being subsequently made to the implantation tool shown in FIGS. 18-21) can be easily attached to the projecting hex portion 30 of the driving worm 18 for actuating the worm 18 and meshing outer beveled worm gears 20/22. The present invention contemplates any bit configuration for rotating the central worm 18, such including without limitation the hex key profile as depicted. It is also advantageous, although not limiting to the present invention, to have the worm gear bit project from the surface of the main jack body in order to enable easier in situ access and adjustment such as following an initial surgical implantation.

Each of the subset body portions 12/14 also depict gripping surfaces configured as inwardly contoured or recessed pockets for receiving the consecutively arranged superior articular processes. A first lower pocket is configured in the lower positioned subset body portion 12 and is defined by a pair spaced apart extending sides or tabs 32/34, each further exhibiting opposing side surface gripping or teethed portions 36/38 and a series of further inside middle gripping locations 40 for configuring a first superior articular process receiving pocket.

As shown, the upper body 16 includes similar upper recessed gripping surfaces forming a pocket defined by a further pair of spaced apart sides 42/44, each further exhibiting opposing side surface gripping teeth 46/48 and additional inside middle gripping locations 50 for configuring a second superior articular process receiving pocket. As described, the spaced apart pairs of sides or tabs 32/34 and 42/44 of the opposite extending gripping portions are adapted to seat upper and lower consecutive superior articular processes.

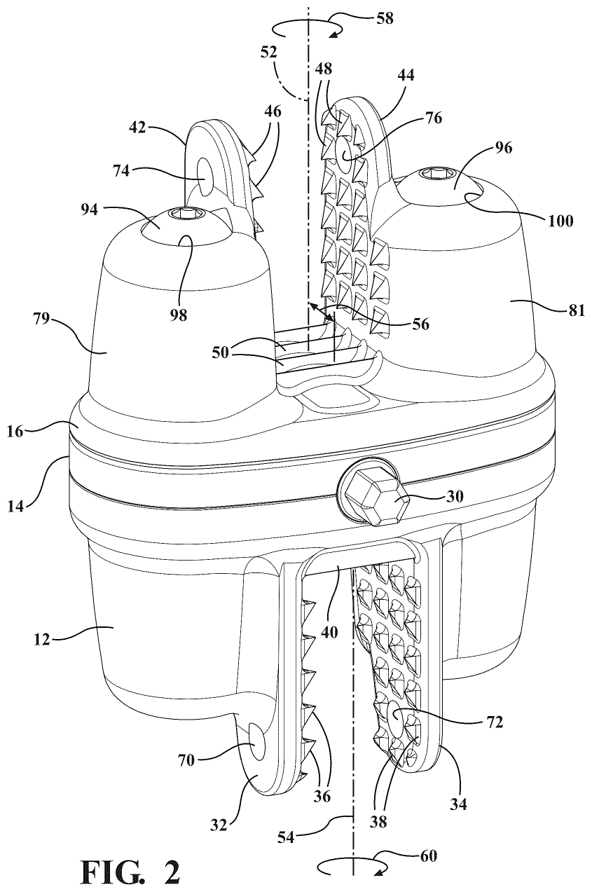

As shown, the arrangement of the inward facing teeth 36/38 configured upon the lower tabs 32/34, along with opposing teeth 46/48 of the upper tabs 42/44 provide for unidirectional insertion of the process within the “U” shaped channels in a manner which prevents reverse withdrawal or detachment. As best shown in the illustration of FIG. 2, a pair of elongated axes 52 and 54 are depicted which extend through axial centerline locations of each of the upper 42/44 and lower 32/34 pairs of tabs.

As further shown, these centerline axes 52/54 are separated by a horizontal depth 56, the significance of which is that inward compressing forces exerted by the superior articular processes (see as further represented in FIG. 2 by upper 58 and lower 60 inward opposing rotational movements) against the upper and lower jack body portions results in an inward rotating movement exerted upon the channel defining pairs of tabs 32/34 and 42/44, with the further result being that detachment of either or both of the processes from the jack bodies or halves is better avoided. In combination, the configuration and arrangement of the tab surface mounted teeth 36/38 and 46/48 contribute to providing additional gripping and retaining resistive engagement against the facet surfaces of the processes.

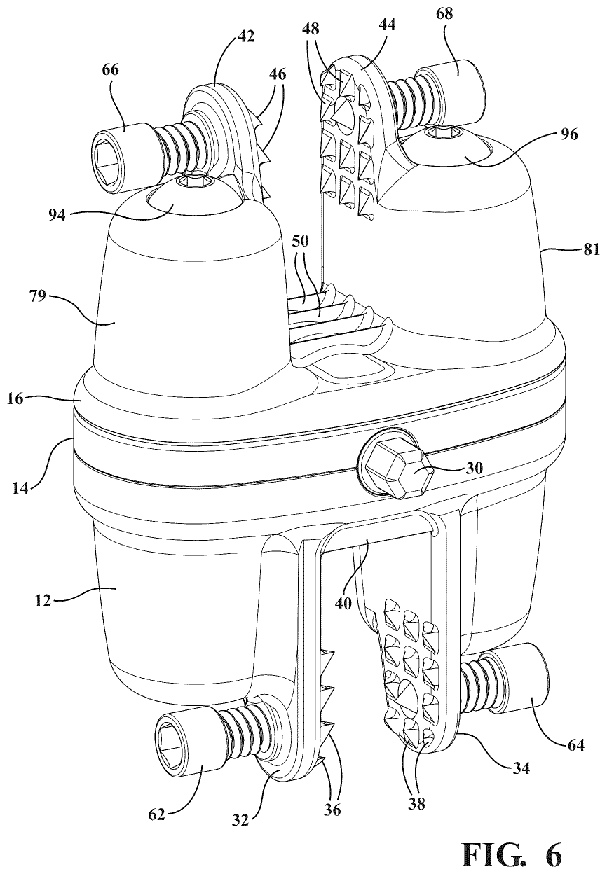

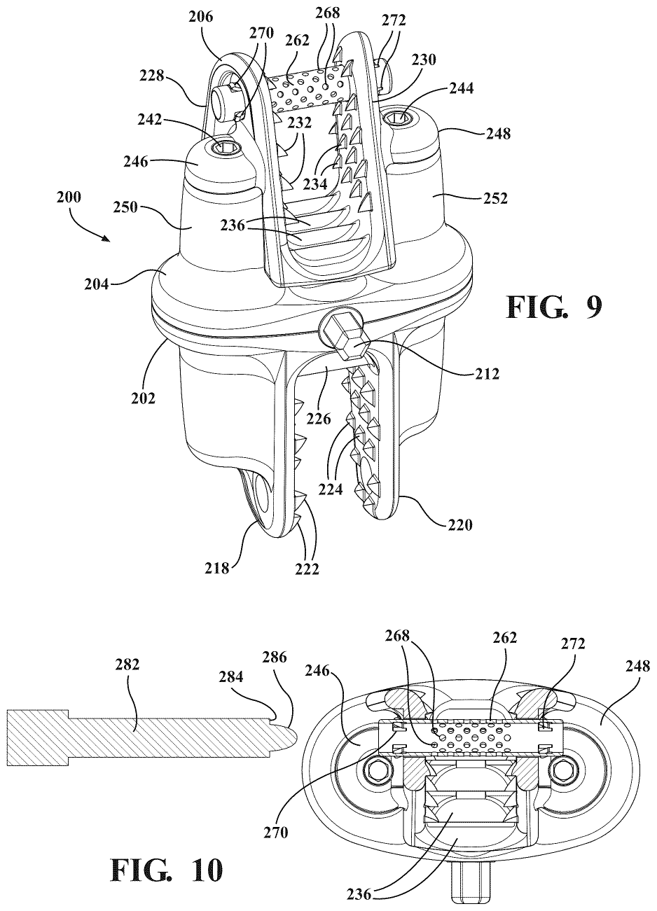

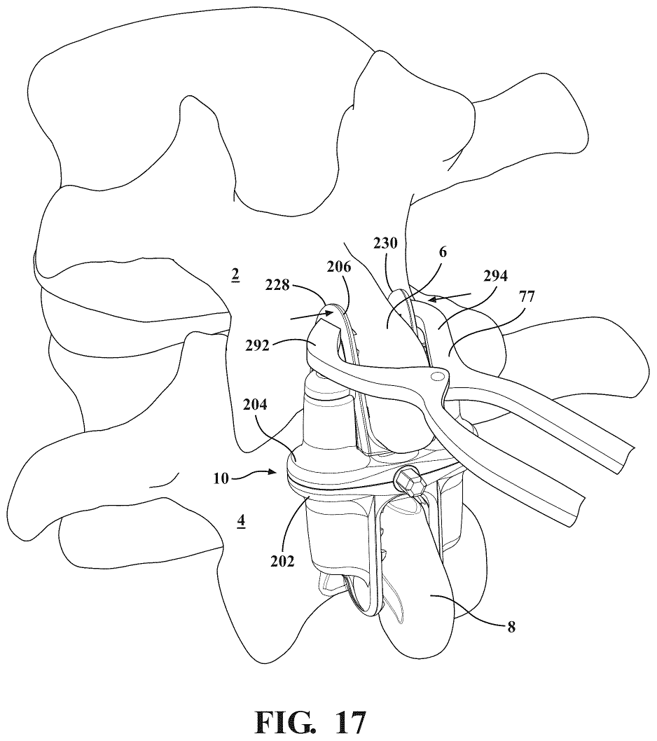

Any type of screw fastener (such as shown by lower pairs 62/64 and upper pairs 66/68 of hex socket head screws in FIG. 6) is employed with each gripping portion and which, upon attaching through pairs of apertures 70/72 and 74/76 formed through the spaced apart pairs of tabs 32/34 and 42/44, provides for anchoring the lower and upper jack halves to the respective vertebral processes. It is further envisioned that alternately configured screws (including a single elongated upper and lower screw drilled through the attached processes and including the tubular shaped rivets depicted in subsequent FIG. 9 et seq.) or push in clips (not shown) can be utilized. Alternatively, the gripping portions defining each pocket can be provided without aligning apertures (see FIG. 1) and which can be crimped into engagement with the vertebral processes 2 and 4, such as without the use of separate screws. This is best depicted in the alternate environmental mounting configuration of FIG. 17 depicting a pair of pliers 77 for inwardly crimping and deforming the pairs of tabs 32/34 and 42/44 to engage the spinous processes 6 and 8 (this with or without the additional use of the mounting fasteners).

As best shown in FIGS. 3-5 and 7, the afore-described elongated stems 78/80 include upper ends anchored to outer superstructure locations 79 and 81 of the upper body portion 16, these in turn nesting over the superstructure portions 15 and 17 of the subset body portion 14 (see as best shown in FIG. 3).

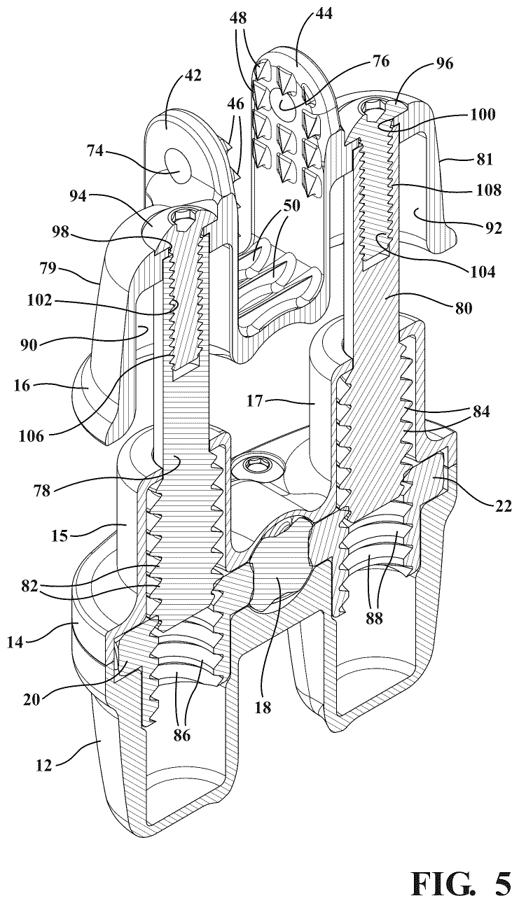

As depicted in one non-limiting embodiment, the stems 78/80 each include outward spiraling threads 82 and 84 which are coaxially interiorly supported within opposing interior threads 86 and 88 associated with the outer bevel gears 20 and 22. The upper ends of the stems 78/80 extend within the hollowed and bell shaped interiors (see at 90 and 92 in the cutaway of FIG. 5) associated with the superstructures 79/81 of the upper jack half body 16.

As best shown in the cutaway views of FIGS. 3 and 5, a pair of screws 94 and 96 can install downwardly through upper end aperture receiving locations 98 and 100 formed in the bell shaped superstructure portions 79/81 of the upper jack half body 16. In one non-limiting option, the upper extending ends of the stems 78/80 have interiors which are open and include threads 102/104 which receive exterior threads 106/108 disposed on the stems of the screws 94/96 in order to elevate the upper jack body 16 (see FIGS. 4-5) in response to rotational actuation of the central worm gear 18 via the projecting hex bit portion 30.

In this fashion, and upon pre-positioning and initial attachment of the spinal jack 10 between the succeeding superior articular processes, an external tool bit (such as again a socket associated with the implantation tool of FIG. 18) engages the projecting hex bit 30 of the central worm gear 18 and further, upon being rotated in a selected rotational direction, results in the outer bevel supported gears 20/22 being rotated. Simultaneously, the inner spiral threads 86/88 of the outer gears 20/22 in turn actuate the inter-engaged threads 82/84 of the stems 78/80 in order to elevate the stems and upper end supported spinal jack body 16.

In this manner, the stems 78/80 are elevated along with the upper supported and process engaging body 16 relative to the assembled subset portions 12/14 of the lower main body. As again best shown in FIG. 5, interior pockets 110/112 can be defined in the lower body portions 12/14 for seating extending displaceable ends of the stems 78/80 in a manner allowing a desired degree of travel relative to the outer gears 20/22 for bi-directional adjustment of the upper spinal jack half 16 relative to the inter-assembled lower spinal jack half 12/14.

Although not shown, it is envisioned and understood that additional spinal braces and the like can be provided (not shown) which can be installed against the lateral processes of each vertebrae and in order to provide additional vertebral support depending upon the nature of the spinal injury being addressed. The construction of the worm gear arrangement of the present invention also provides the ability to make minute or fine incremental adjustments to the jack bodies, and without the requirement of implementing further anti-reverse motion locking mechanisms for preventing inadvertent reverse inward adjustment in response to compressive forces exerted by the spinal processes on the jack halves.

Accordingly, the present invention provides a number of unique features not present in other competing spinal jack devices. These include modifying the gripping teeth design beyond those depicted in the attached illustrations and which can include any alternate design, pattern, facet arrangement or the like for facilitating unidirectional (slide in) insertion, along with frictional resistance to reverse direction withdrawal or disengagement of the jack from the superior articular processes.

Beyond the protruding hex bit portion illustrated, it is further understood and anticipated that other bit engaging profiles can be provided for receiving a suitable adjustment tool, such as post initial implantation within the patient and during a subsequent adjustment of a spatial distance between the succeeding articular processes supported by the jack. Other features include any arrangement of side screws or pushpins, such as two or four, provided in any style or amount.

The use of a worm and worm gear design is also understood to prevent inadvertent or reverse inward adjustment of the jack halves, such as in response to compressive forces exerted between the articular processes. In this fashion, the worm/worm gear jack design of the present invention can be optionally provided without the need for additional restraining or locking mechanisms for preventing undesirable compressive adjustment in situ within the patient.

Other features include the stepped or depth offsetting design of the oppositely directed upper and lower jack bodies which again create inwardly directed moment forces in response to compression by the spinal processes on the attached jack. In this fashion, the “U” shaped design of the jack halves is caused to inwardly compress or tighten in response to the compressive applied loads, and as opposed to outward directed momentum forces which would tend to detach the jack halves from the attached spinal processes.

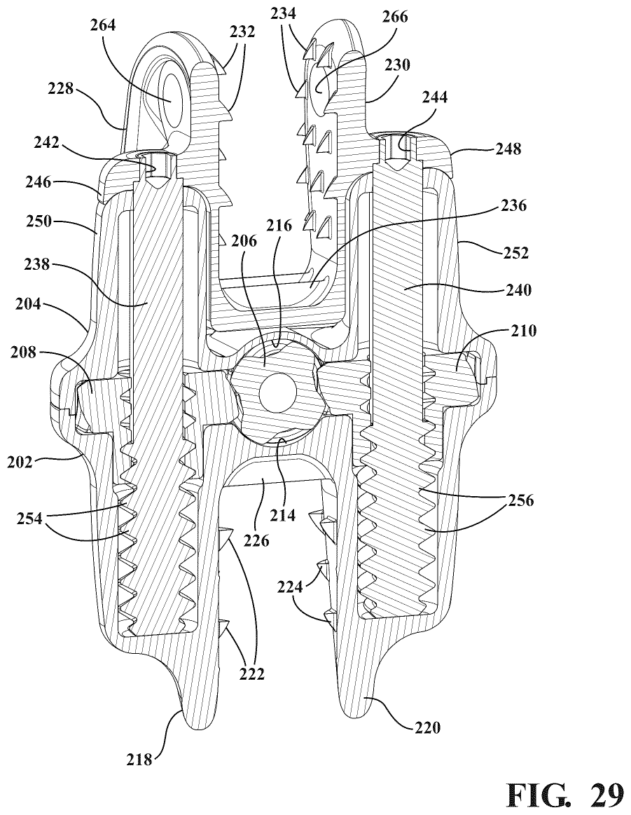

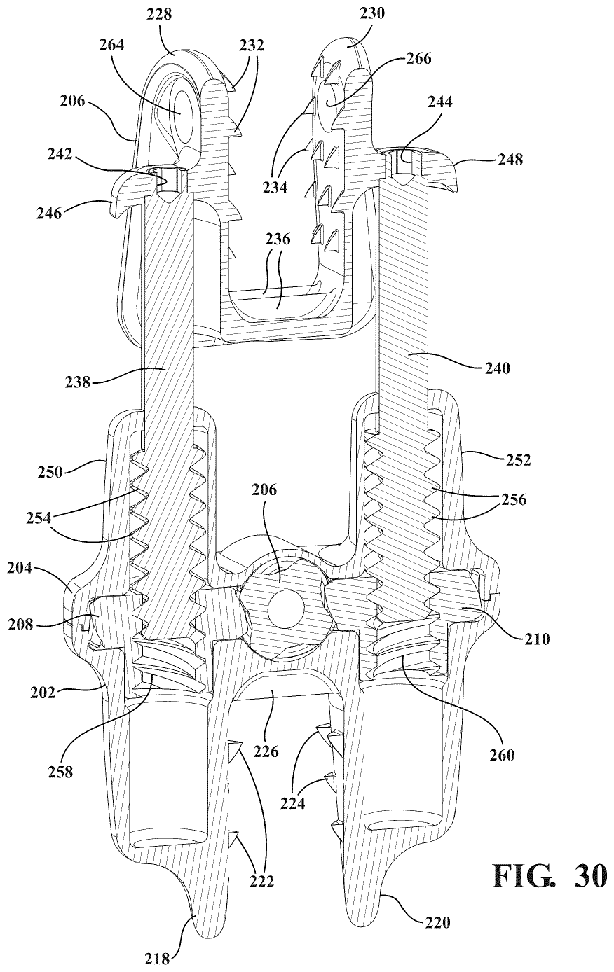

Proceeding now to FIG. 9, a perspective view is generally shown at 200 of a spinal worm screw jack according to a further non-limiting variant and again depicts the first and second body portions shown in a retracted position. As further best shown in the cutaway views of FIGS. 29-30, the lower or main body is reconfigured as subset body portions 202 and 204, along with a redesigned upper body portion 206.

The subset lower body portions 202/204 and upper displace-able body portions 206 are again each constructed of a suitable sanitary medical grade material not limited to any of a metal (e.g. typically titanium) or other plastic composition. The subset assembled lower body portions 202/204 can again be sonic welded or otherwise secured to define a package interior space for collectively seating a worm gear mechanism, further including each of a central worm 206 (see also FIGS. 1-7) and inter-engaging and rotationally actuated outer located gears 208 and 210 (again FIGS. 29-30). The outer gears 208/210 are likewise arranged so that a rotational centerline axis of each is arranged in a perpendicular direction relative to a rotational centerline of the central worm 206.

The central worm 206 again includes a spiral array of gear teeth (not shown in the variant of FIG. 9 with reference again being made to 24 in FIG. 7) extending along its generally horizontal length which mesh with the crosswise arranged and annular arrayed gear teeth, again previously referenced at 26 arranged upon each of the outer worm gears 208/210 which are arrayed on opposite sides of the central worm 206.

A projecting hex bit portion 212 is integrally formed with an axial end of the central worm 206 and which seats within and (optionally) projects forwardly from aligning annular rim portions (see at 214 and 216 in FIG. 29 cutaway) defined between the opposing end faces of the subset body portions 202/214.

As with the prior embodiment, each of the subset body portions 202/204 again depict gripping surfaces configured as inwardly contoured or recessed pockets for receiving the consecutively arranged superior articular processes (see again FIG. 8). A first lower pocket is configured in the lower positioned subset body portion 202 and is defined by a pair spaced apart extending sides or tabs 218/220, each further exhibiting opposing side surface gripping or teethed portions 222/224 and a series of further inside middle gripping locations 226 for configuring a first superior articular process receiving pocket.

As shown, the upper body 204 includes similar upper recessed gripping surfaces forming a pocket defined by a further pair of spaced apart sides 228/230, each further exhibiting opposing side surface gripping teeth 232/234 and additional inside middle gripping locations 236 for configuring a second superior articular process receiving pocket. As described, the spaced apart pairs of sides or tabs 218/220 and 228/230 of the opposite extending gripping portions are adapted to seat upper and lower consecutive superior articular processes.

Elongated stems 238 and 240 are again provided (see again FIGS. 29-30) and include upper ends 242/244 anchored to outer support locations 246/248 of the upper displaceable body portion 206, these in turn seating over superstructure portions 250 and 252 of the subset body portion 204.

As previously described in the initial embodiment of FIGS. 1-8, stems 238/240 each include outward spiraling threads 254 and 256 which are coaxially interiorly supported within opposing interior threads 258 and 260 (FIG. 30) associated with the outer bevel gears 208 and 210. In this fashion, rotation of the hex bit 212 elevates the upper jack body 206 relative to the lower jack subset body portions 202/204 in like fashion as previously described in the first embodiment 10.

FIG. 9 depicts a tubular mounting rivet style fastener 262 according to one non-limited variant of the invention which can be mounted through a pre-drilled hole through the spinous process aligning with a pair of apertures (such as at 264/266 in FIGS. 29-30) formed in either of the upper 228/230 or lower 218/220 gripping pocket defining tabs. The mounting rivet 262 can be solid or, as shown, hollow in a tubular fashion with bone in-growth apertures 268 distribute across the width and circumference of the tubular shaped body. Locating and engaging embossments 270 and 272 are formed at width spaced and circumferentially arrayed locations which, upon width directed installation, align with and simultaneously seat against opposite outward rim surfaces of the mounting tables (again at 228/230) in order to define a correct installation position (again best shown in FIG. 9).

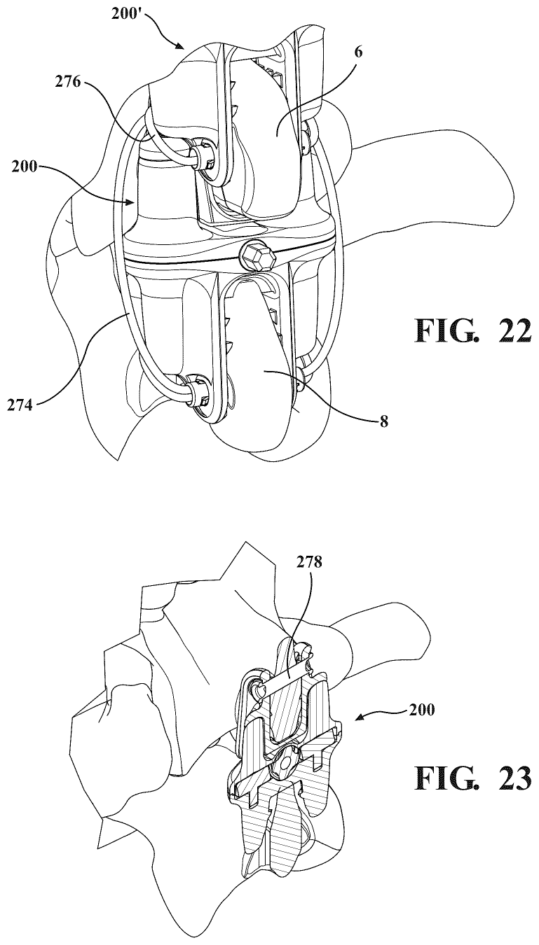

FIG. 22 depicts a pair of implanted spinal jacks 200 and 200', such as individually shown in FIG. 9, with the further installation of retention cables, see at 274 and 276 extending through the hollowed interior of the tubular rivets 262 for providing additional retention properties.

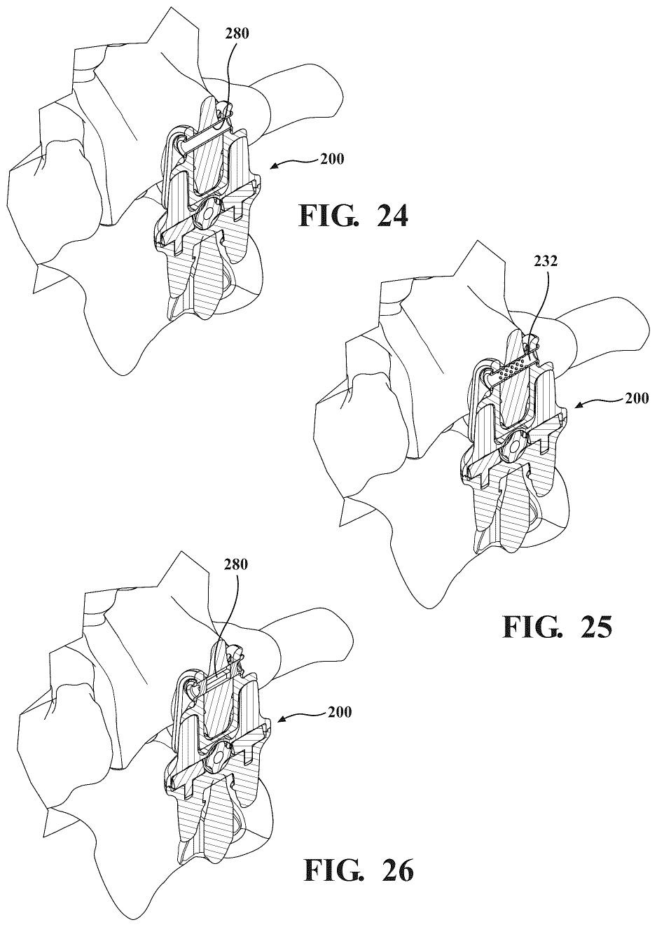

FIG. 23 presents a vertical cutaway of a selected implanted spinal implant or jack such as shown in FIG. 22 and exhibiting a solid tubular mounting rivet 278. FIG. 24 is a similar view to FIG. 23 and depicting the alternate use of a sold/non-apertured tubular mounting rivet, with FIG. 25 again depicting the hollowed tubular mounting rivet 262 with bone in-growth apertures 268 such as depicted in FIG. 9 et seq. FIG. 26 provides a yet further succeeding view depicting a modified tubular rivet 280, including solid end portions and partially open middle area.

Referencing now FIG. 10, an overhead and partially cutaway depiction of the spinal jack 200 of FIG. 9 is shown with a width directed rivet removal tool bit 282 prior to seating and laterally displacing the illustrated hollow tubular rivet 262 from between the gripping pocket tabs 228/230 and the selected gripped spinous process. The removal tool bit 282 includes an elongated body of selected diameter with a forward abutment shoulder 284 and terminating conical forward end 286.

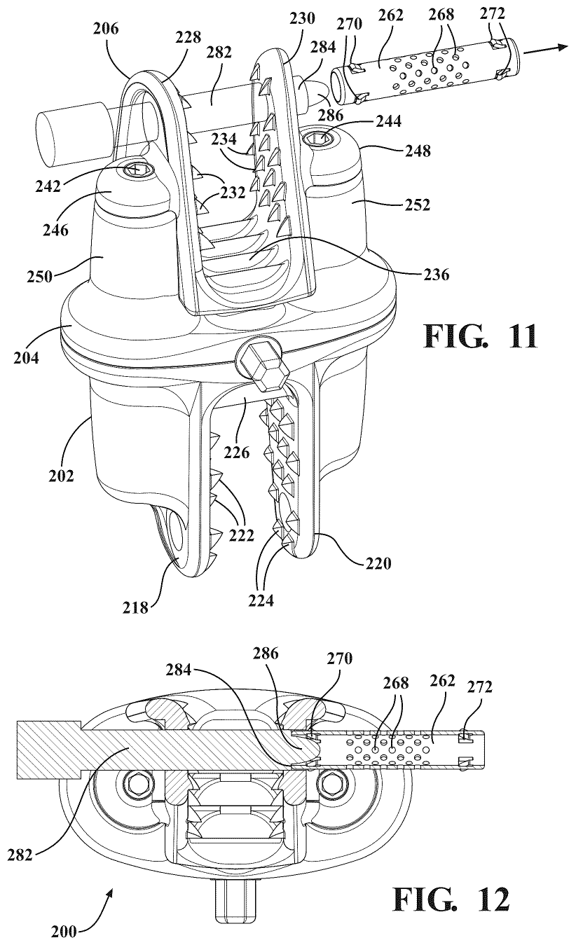

FIG. 11 presents a succeeding view to FIG. 10 and depicting in perspective the removal of the tubular shaped rivet 262, with FIG. 12 providing a similar view to FIG. 10 and depicting the rivet 262 in a substantially removed position in which the width directed tool bit 282 is substantially translated between the gripping tabs 228/230 in a fashion which permits the forward abutment shoulder 284 to seat against the opposing tubular end of the rivet 262, with the conical forward end 286 seating within the rivet.

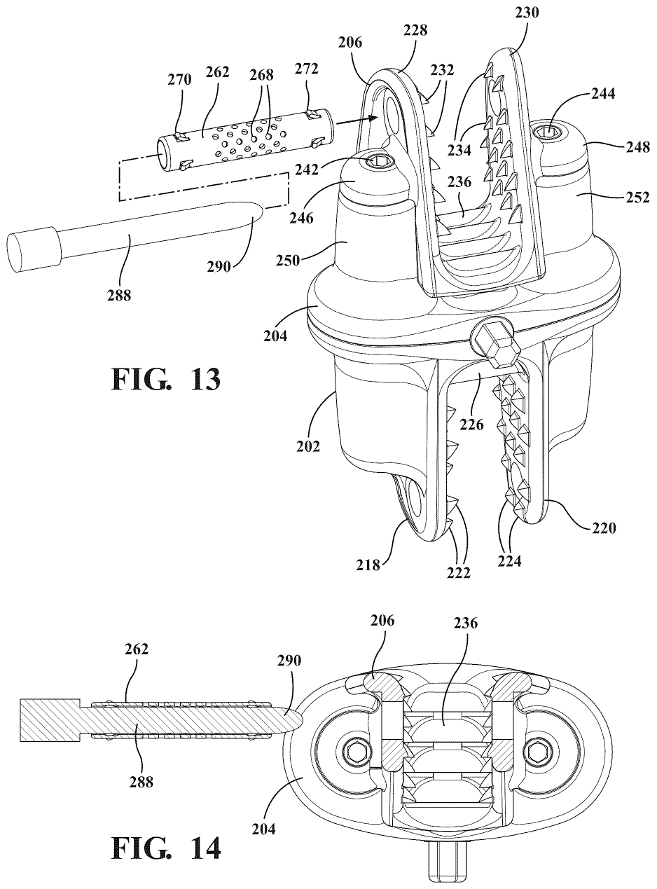

FIG. 13 is an illustration similar to FIG. 9 and depicting the tubular mounting rivet 262 in a pre-installation position, along with an alternately configured width directed rivet installation tool bit 288 of a marginally smaller diameter as opposed to the removal bit 282 and which, as shown in FIG. 14, inserting through the hollow interior of the rivet 262 such that a forward end 290 of the tool projects beyond the rivet. As previously described, the rivet 262 depicts engagement embossments 270/272 configured upon the exterior circumference of the tubular rivet in order to define a correct lateral mounting position shown in FIG. 9.

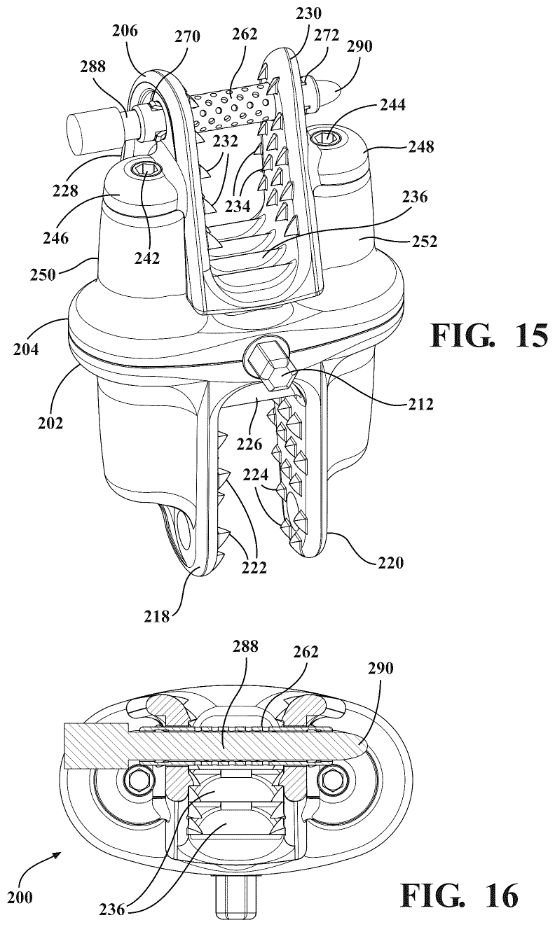

FIG. 14 is an overhead view similar to FIGS. 10 and 12 and, progressing from FIG. 13, depicts the pre-installed tubular rivet seated over the width directed installation tool bit 288 prior to inserting installation. FIG. 15 is a substantial repeat of FIG. 9, showing the tubular shaped mounting rivet engaged and prior to retraction of the width directed installation tool. FIG. 16 provides an overhead and partially cutaway view of FIG. 15, similar in presentation to each of FIGS. 10, 12, and 14, and again showing the selected rivet fastener 262 installed through the pre-drilled passageway formed in the vertebral process.

Proceeding to FIG. 17, an environmental illustration similar to that shown in FIG. 8 depicts a crimping option including a plier's like tool pivotally associated and opposing jaws 292/294 of the crimping tool 77 for deforming and affixing the extending tabs (see again at 228/230) defining each of the upper and lower gripping pockets against the spinous processes, such as additionally or alternative to the use of mounting fasteners of FIG. 9.

FIG. 18 provides a perspective of an installation tool 296 which provides for locating and resistive seating of the spinal implant 200 against the spinous processes of the successive vertebrae (see again FIG. 8) such that, and upon initial linear seating of the implant pockets to the spinal or spinous processes, exerted impact forces upon the tool resistively seat the gripping pockets against the spinal processes. In the initial installation configuration, a rectangular forward end location 298 of the tool 296 is biasingly compressed between the opposing undersides of the process gripping pockets in a partially separated configuration in order to permit extension and initial affixation to the spinous processes within the body cavity.

The implant tool 296 includes an elongated neck 300 which extends from the forward rectangular end location 298 to a rear annularly expanded support collar 301 which in turn support a removable handle 302. Upon removal of the handle 302, it reveals a rear projecting end 304 of a tool bit driver extending within the tool interior of the neck 300 to a forward located bit engaging socket (not shown) which is located within the forward interior of the rectangular end 298 and which, as arrayed in FIG. 18, is in turn engaged to the receiving bit (30 or 212) of the implant jack. Successive rotation of the rear driver 304 rotates the hex bit to initiate separation of the upper and lower implant bodies.

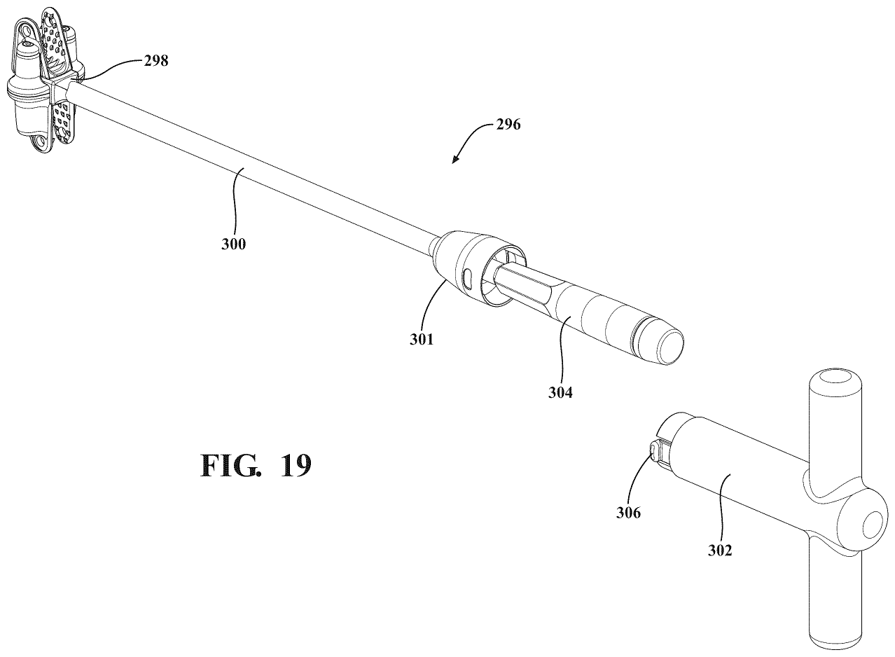

FIG. 19 is a succeeding view to FIG. 18 and depicting the removal of the rear handle end 302 of the tool, again revealing the rear projecting end of the tool bit driver 304 extending within the tool interior to the forward located bit engaging socket. Removal of the handle 302 can be facilitated by press tabs 306 which seats through a matching recess 308 in the support collar 301 and which, upon being inwardly depressed, allows the handle to removed, such as following an initial forward impact assisted installation of the implant 200, such as with the assistance of a mallet, hammer or the like.

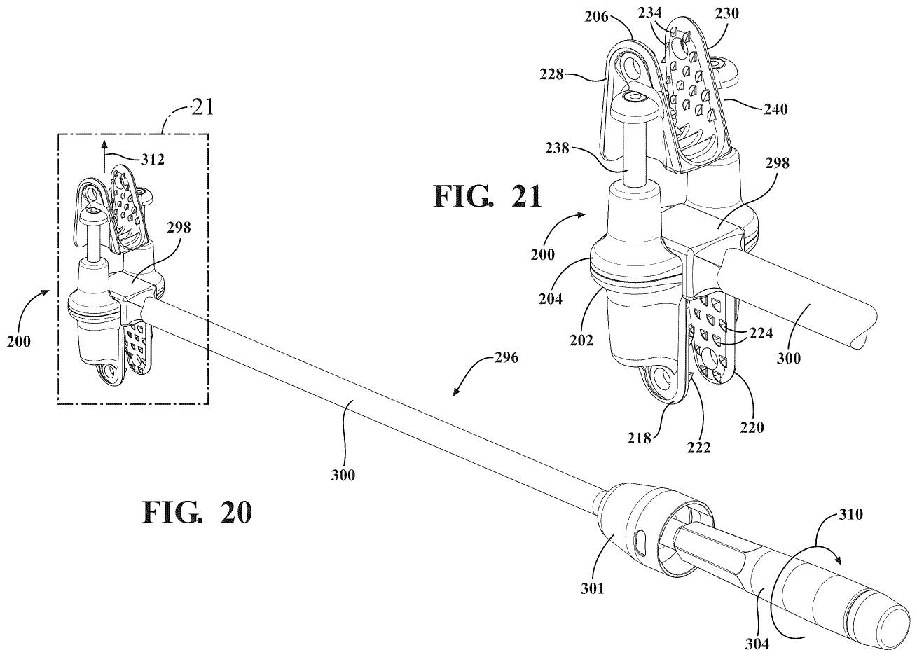

FIG. 20 is a further succeeding view to FIG. 19 and depicts a rotation (see arrow 310) of the rear driver 304 in order to rotate the hex bit to initiate separation of the upper and lower implant bodies (see as further referenced by upward directional arrow 312). FIG. 21 is an enlarged view of forward area 21 of the implant tool depicted in FIG. 20 and depicting the rotating expansion of the upper and lower spinal body portions (at this point the initial lodging of the implant gripping pockets to the spinous processes as shown in FIG. 8 allowing for continued connection between the hex bit driver of the tool with the implant hex bit 212) following upward displacement of the upper body portion 206 away from compressing abutment with the rectangular forward location 298.

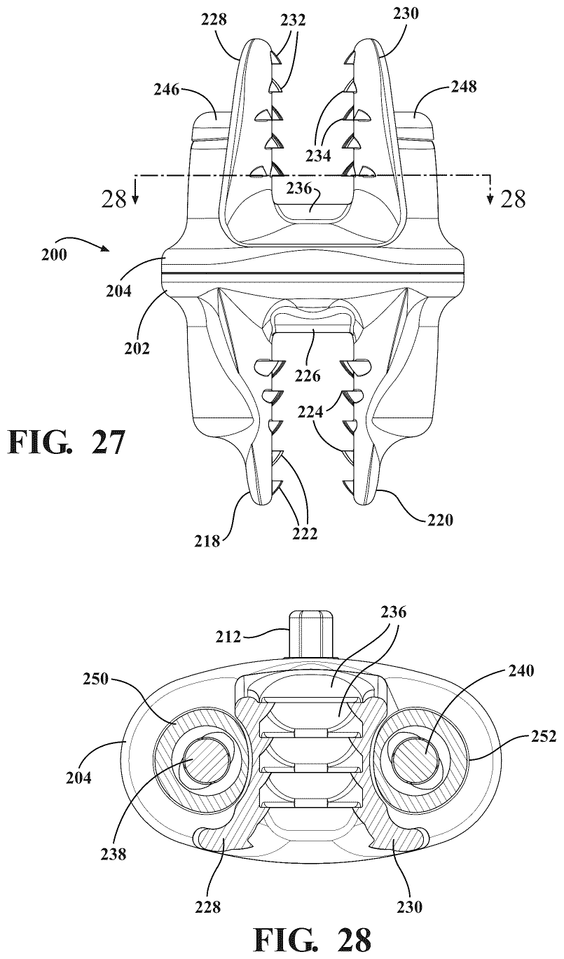

Referring to FIG. 27, a rear plan view is shown of selected spinal implant jack 200 and depicting an arrangement of the gripping teeth associated with each of the process engaging pockets configured in each of the upper and lower body portions. As generally illustrated, the gripping teeth patterns are sized smaller to larger in each of inward and rearward engaging directions against and around the spinous processes. FIG. 28 provides a horizontal cutaway view taken along line 28-28 of FIG. 27 and better illustrating the configuration of the base surface gripping teeth associated with the upper process engaging pocket.

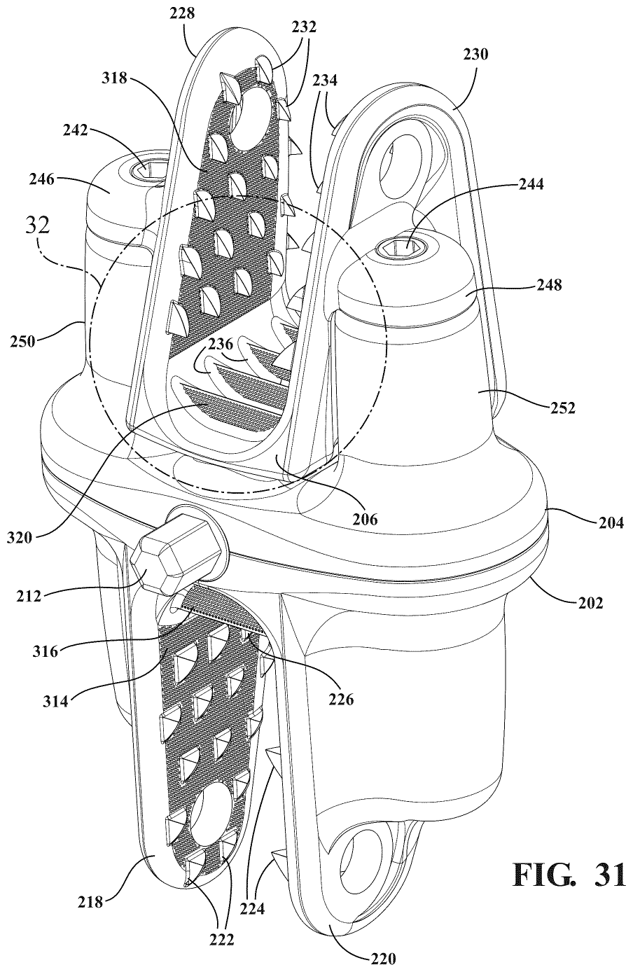

Proceeding to FIG. 31, presented is a depiction of a spinal implant jack exhibiting surface aperture (also termed “latticed”) patterns distributed across the gripping pockets. These are generally shown by latticing patterns 314 and 316 visible in lower pocket along gripping locations 232 and 236, with like upper pocket latticing patterns at 318 and 320 corresponding with the arrangement of gripping locations 232 and 236.

Surface latticing can be accomplished as part of a three dimensional or additive printing process for forming each of the individual outer body components, including each of the first 202 and second 204 subset lower body portions and displaceable upper body 206, without limitation the additive printed material including a titanium or other suitable medical grade material including other metals or polymeric composites.

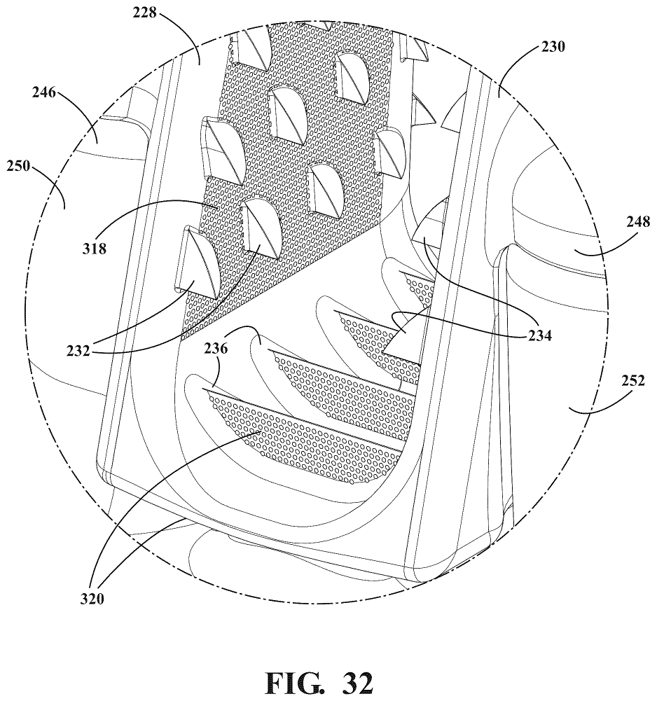

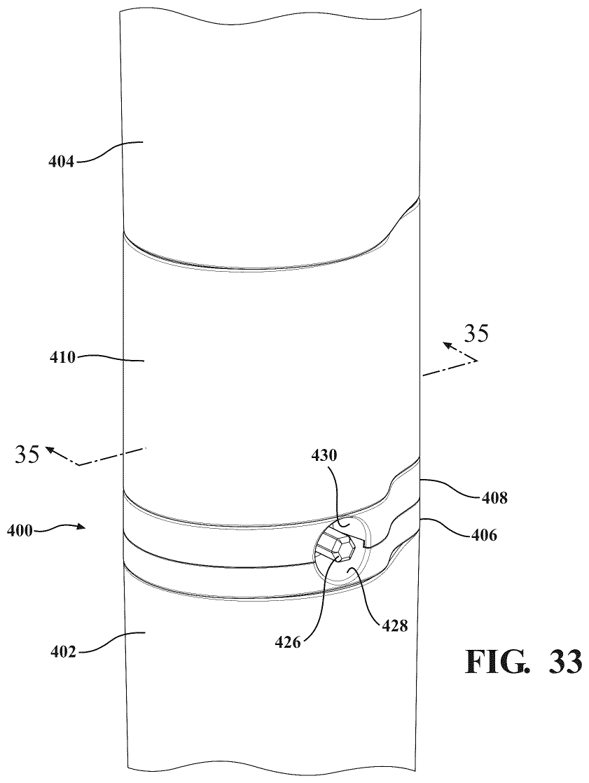

FIG. 32 illustrates an expanded area referenced in FIG. 31 of the spinal process gripping pocket associated with the upper body portion and better depicting the latticing of the surface layers in order to promote bone in-growth following implantation. The additive printing techniques employed for producing the implant body components permit the configuration of the teethed gripping portions according to any desired varying sizes and directions, such as again increasing in size in both inward and/or downward seating directions in order to enhance the initial seating engagement of the spinal processes into the implant gripping pockets;

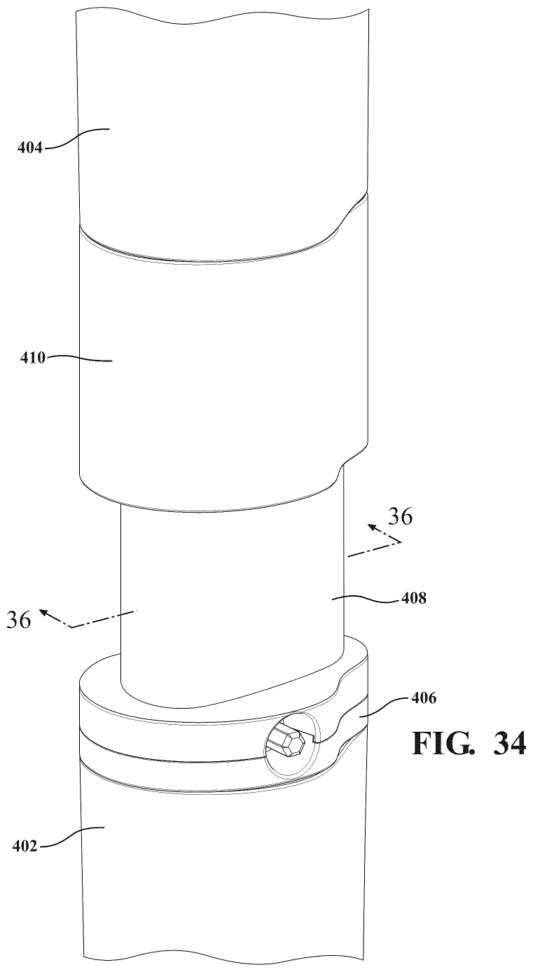

Proceeding to FIG. 33, presented is a perspective environmental 400 of a further version of an implant for non-vertebral applications, such as in use with first 402 and second 404 segmented bones associated with any of a humerus, femur or the like. FIG. 34 presents a succeeding illustration to FIG. 33 in which lower body implant portions 406 and 408 and upper body implant portions 410 are shown in an expanded configuration.

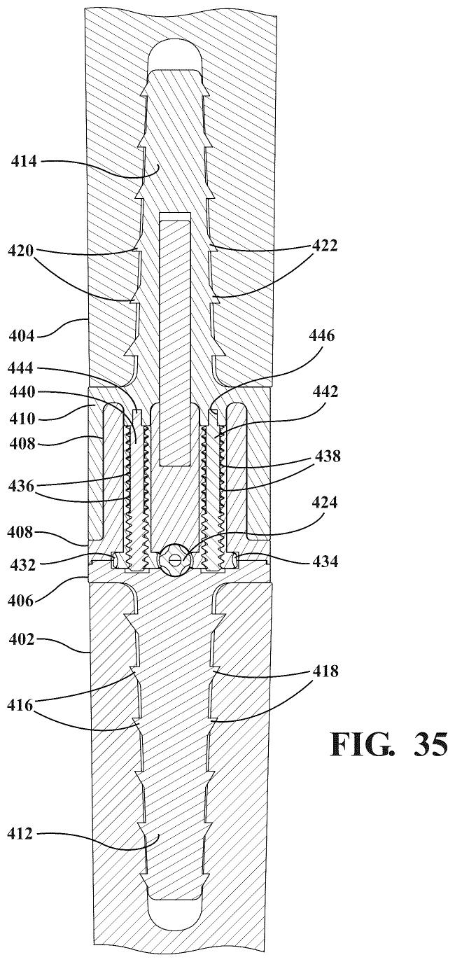

FIG. 35 is a vertical cutaway taken along line 35-35 of FIG. 33 and depicting the configuration of the implant in which the U shaped vertebral process seating pockets of the earlier embodiments are reconfigured as inner elongated body portions, including lower elongated body portion 412 integrated into lower implant 406 and upper elongated body portion 414 integrated into upper body 410.

The body portions 412 and 414 each further exhibit integrated gripping portions (see at 416/418 for lower elongated portion 412 and at 420/422 for upper elongated portion 414) which seat within the marrow interiors of the elongated bone segments 402/404, for bonding the upper 410 and lower 406/408 body portions to the opposing end faces of the bone segments. As further shown, the upper 408 of the lower body portions and the nesting upper body portion 410 each exhibit a multi-walled and nesting arrangement for providing effective multi-directional support in any loading direction.

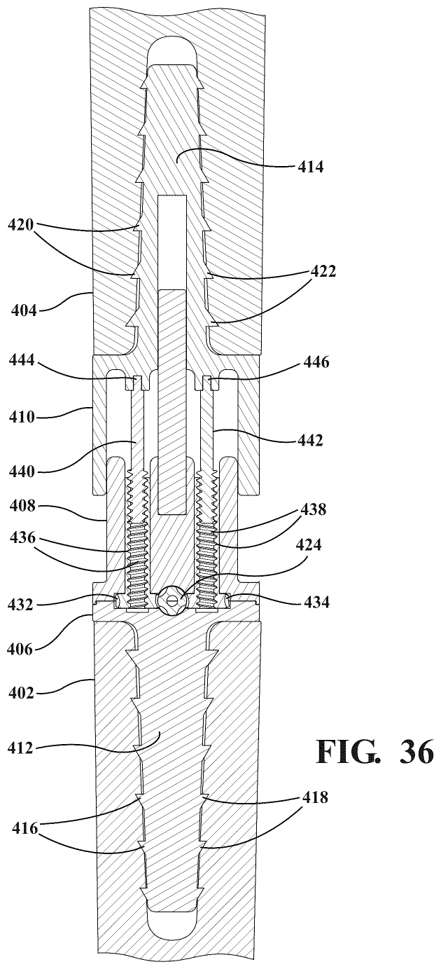

FIG. 36 is a vertical cutaway taken along line 36-36 of FIG. 34 and depicts the expanded configuration established between the implant upper 410 and lower 406/408 body portions for establishing a desired and adjustable separation distance between the bone segments 402/404. The body portions 406/408/410 can again each be constructed by any forming process including molding, additive manufacturing or the like.

As with the spinal implant bodies 10 and 200, the lower body portions 406/408 contain a worm gear mechanism including a central worm gear 424 with surface accessible integrated hex bit 426 recessed into a surrounding pocket defined by mating rim surfaces 428 and 430 defined in body portions 406/408. FIGS. 35 and 36 depict outer gears 432/434 which seat lower threaded ends 436/438 of a pair of displacement stems 440/442. The stems are further connected at upper ends 444/446 to inside locations of the upper body portion 410.

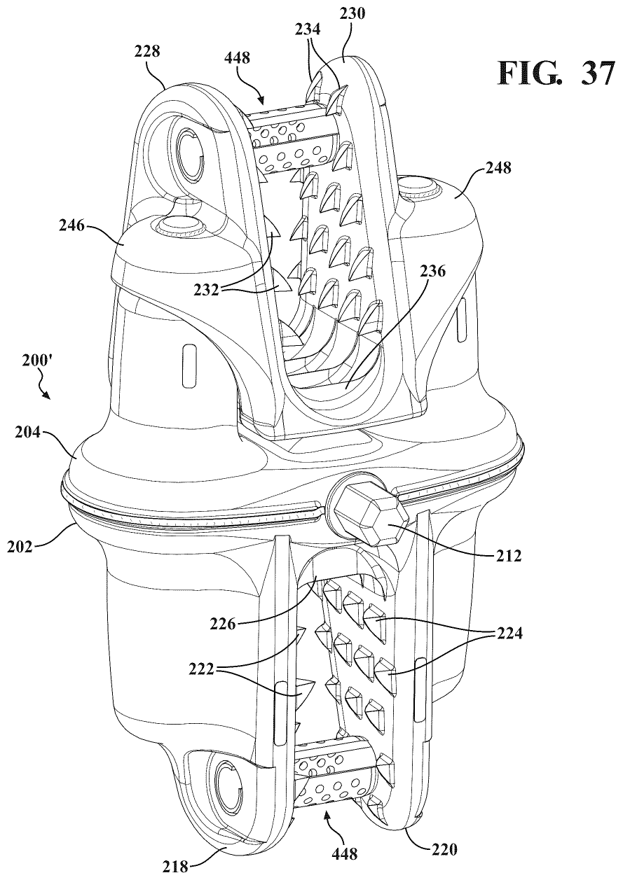

FIG. 37 presents a perspective view of a spinal worm screw jack, generally at 200′, similar to that previously shown in FIG. 27 and according to a further non-limiting variant and having upper 204 and lower 202 body portions shown in a retracted position prior to being installed between succeeding superior articular processes associated with upper and lower consecutive spinal vertebra. Similar elements to those previously shown in FIG. 27 are likewise numbered such that a repetitive description is unnecessary.

Also depicted are split rivets 448 (see also FIGS. 54 and 55) for securing between the aligning apertures formed in spaced apart and gripping pocket defining tabs (see for example lower apertures 70/72 and upper apertures 74/76 in FIG. 1) associated with the spinal jack bodies. FIG. 54 presents a perspective view of the split rivet 448 according to the present invention as initially shown in FIG. 37 and depicting tubular shaped body which is split along its extending length (see opposing split edges 450/452) and including apertures (see as depicted by multiple inner closed rim edges 454) distributed across a width and circumference thereof for facilitating bone in-growth through the apertures.

Without limitation, the construction of the rivets 448 can without limitation be constructed from a machined medical grade titanium or the like which can be bent or otherwise mechanically fashioned so that the rivets provide a desired degree of flex or bend concurrent with being installed within the vertebral bone. As further shown in the rotated plan view of FIG. 55, the split rivet of FIG. 54 shows a slightly enlarged diameter at a central or midpoint location (at 456) of the rivet, this in comparison to the first 458 and second 460 end located diameters. The slight enlargement of the central diameter operates in combination with the flex/expansion of the rivet during its installation into the bone to prevent subsequent disengagement and, as such, does not require separate retaining structure such as shown in the rivet design 262 of FIG. 9 et seq. and including the end locating embossments 270/272.

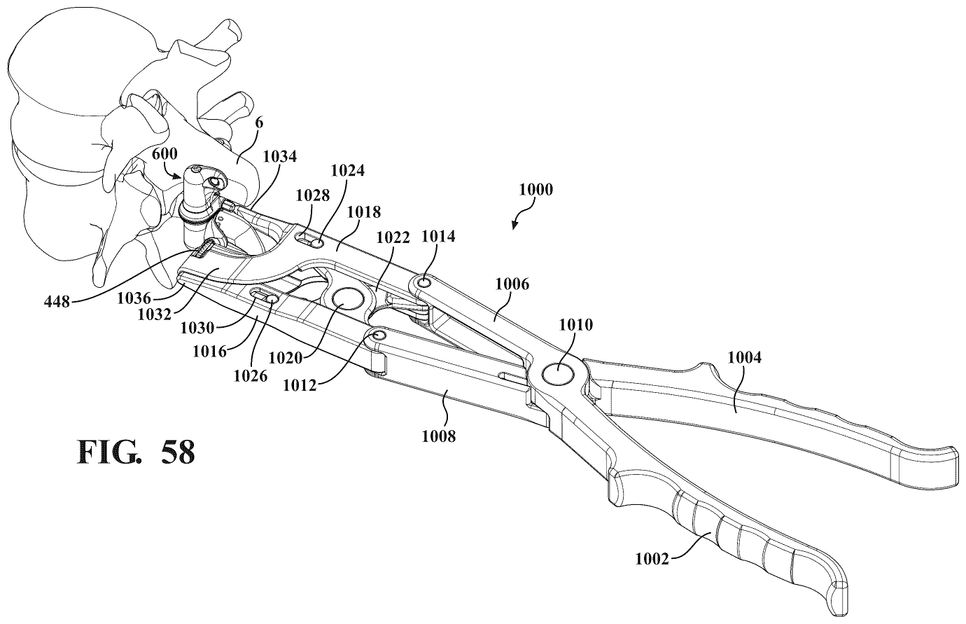

As will be described with further reference to the tool installation protocols of the present invention, the rivets 448 are installed by an associated fixation tool (see as subsequently described in FIG. 58) between the respective upper and lower spaced pairs of apertures within the vertebral bone of each spinous process.

As further noted, the annular rim edges defining the end locations 458/460 of the split rivet are configured as sharpened blade edges which facilitate the split rivet being forcibly pressed through the spinal process bone without the requirement of pre-drilling. Without limitation, both opposite rim blade edges permit the rivet to be loaded within the fixation tool and subsequently driven through the bone from either edge.

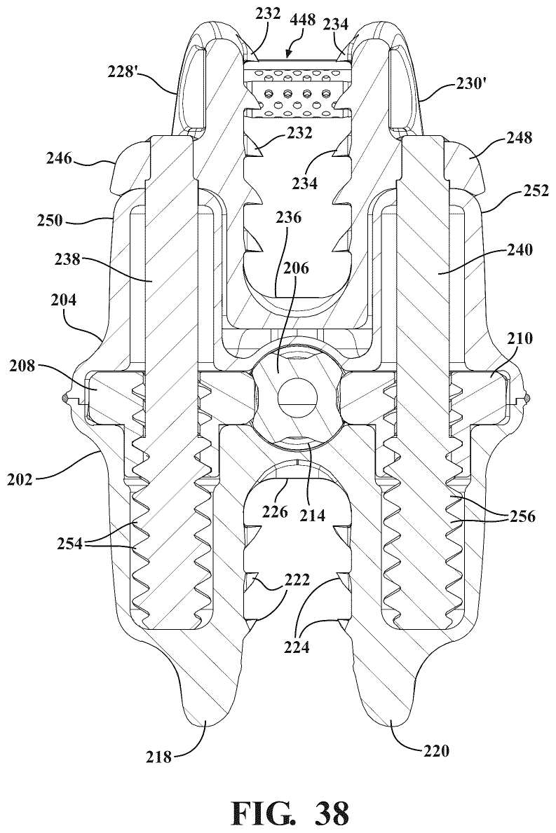

FIG. 38 presents a further perspective and vertical cutaway of the spinal implant jack of FIG. 37, similar to that previously shown in FIG. 29, and according to a non-limiting variant of the present invention in a fully closed position. Similar reference numbers are listed for corresponding features shown in FIG. 29 and including presenting the non-limiting configuration of the worm gear arrangement including the central gear 206, outer gears 208/210 and lift screws 238/240 for elevating the upper spinal body 204 relative to the lower body 202.

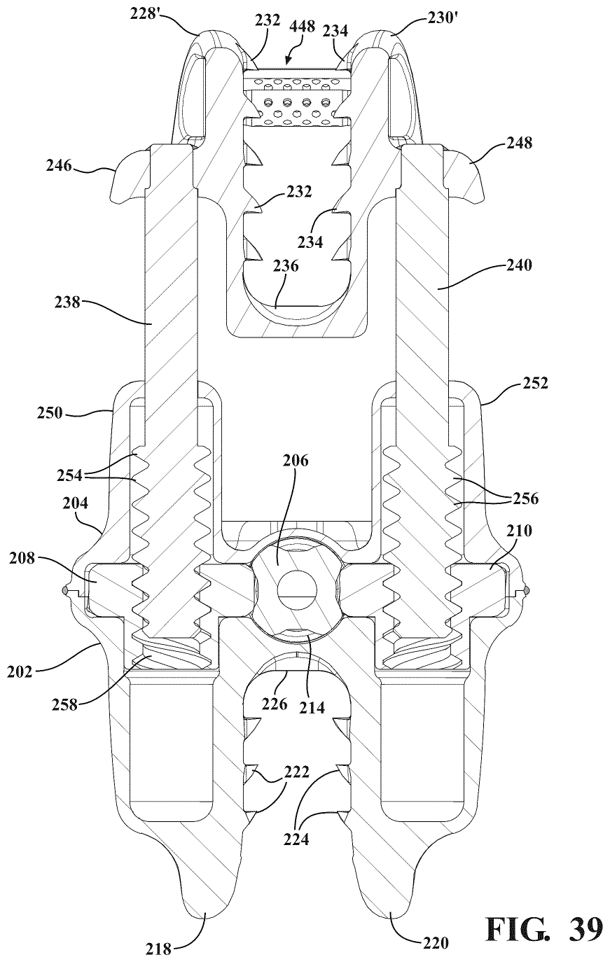

FIG. 39 presents a similar view to FIG. 38, and showing the spinal implant jack in an expanded position similar to FIG. 30. As will be described with reference to the various design configurations for the spinal jack, it is understood that the upper and lower spinal body housing shapes can be varied from any of these depicted herein, along with a range of variations in the central worm gear, outer worm gears and inter-engaging lift screws, thereby facilitating multiple variations for lifting or separating the jack halves with respect to one another.

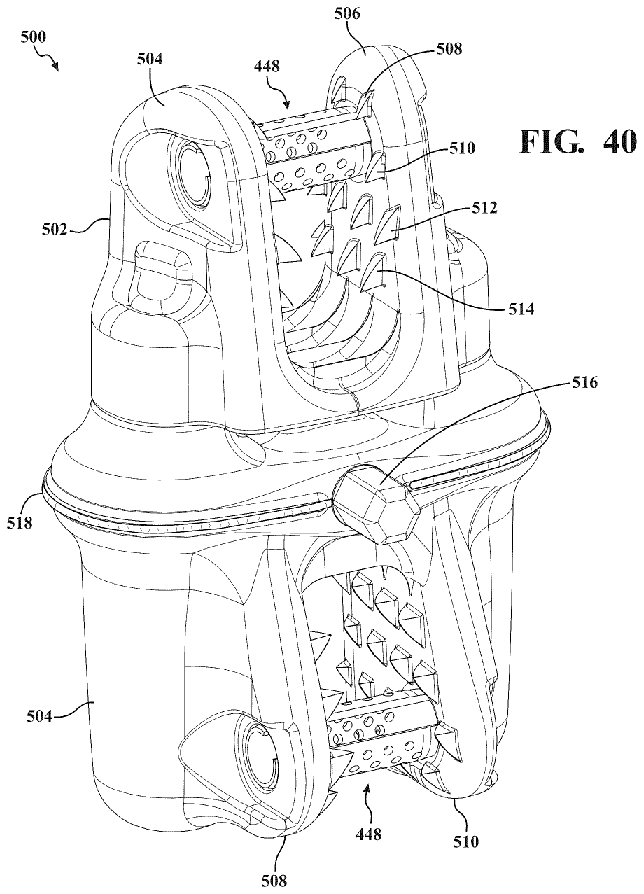

FIG. 40 presents a perspective view of a further non-limiting variant, generally at 500, of a spinal worm screw jack and again having upper 502 and lower 504 body portions shown in a retracted position prior to being installed between succeeding superior articular processes associated with upper and lower consecutive spinal vertebra.

The split rivets 448 are again shown for securing between the upper and lower pairs of aligning apertures formed in the upper (504/506) and lower (508/510) pairs of spaced apart tabs defining the upper and lower “U” shaped gripping pocket of the upper and lower spinal jack bodies. As further previously described, the rivets 448 exhibit bone in-growth promoting apertures when engaged through the spinal vertebrae process. Also depicted are rows of gripping teeth shown in successively increasing size (see by non-limiting example at 508, 510, 512 and 514 in for gripping pocket defined in upper body 502).

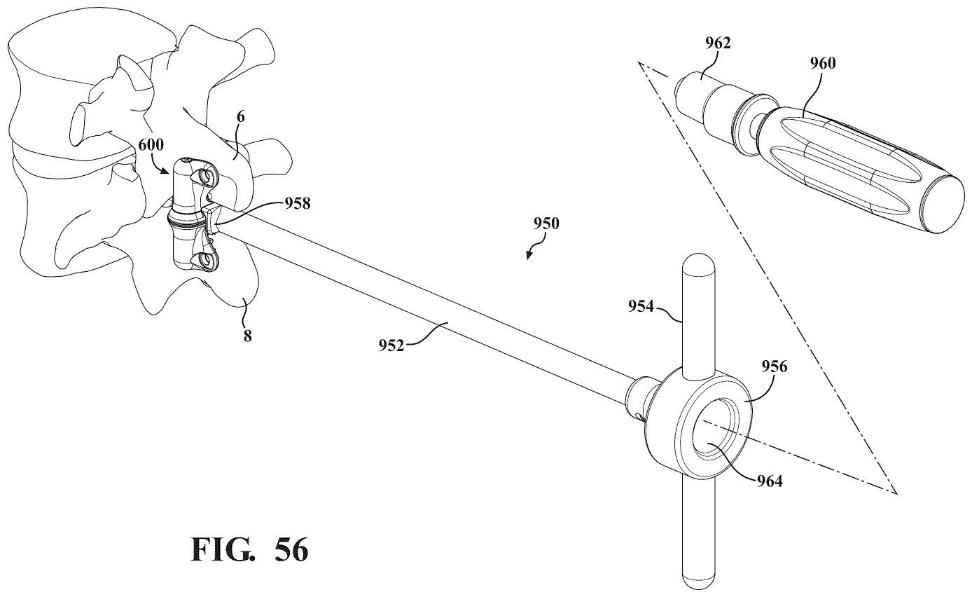

As previously noted, the increasing dimension of each rows of gripping portions is provided so that the succeeding rows can bit deeper into the vertebral bone during installation in order to maximize the gripping aspect of the spinous processes against the pocket apart from the securing force provided by the split rivets. 448. Depending upon the installation orientation of the spinal jack gripping pockets, it is also envisioned that the rows of gripping portions can increase in either of horizontally arrayed rows (as shown) as well as alternately in crosswise vertical rows, and such as to optimize the implantation protocols shown by tool of FIG. 56.

Projecting hex bit 516 is shown and which corresponds to that previously depicted at 212 which is formed integrally with an axial end of the central worm gear. Also depicted are laser weldment locations (see at 518) which seal together the upper 502 and lower 504 spinal body halves, these again including interior cavities which contain the worm gearing components, again including the arrangement of lift screws). As previously described, the components of the spinal jack can be three dimensionally printed from a medical grade Titanium and, upon assembled together, are laser welded or otherwise enclosed.

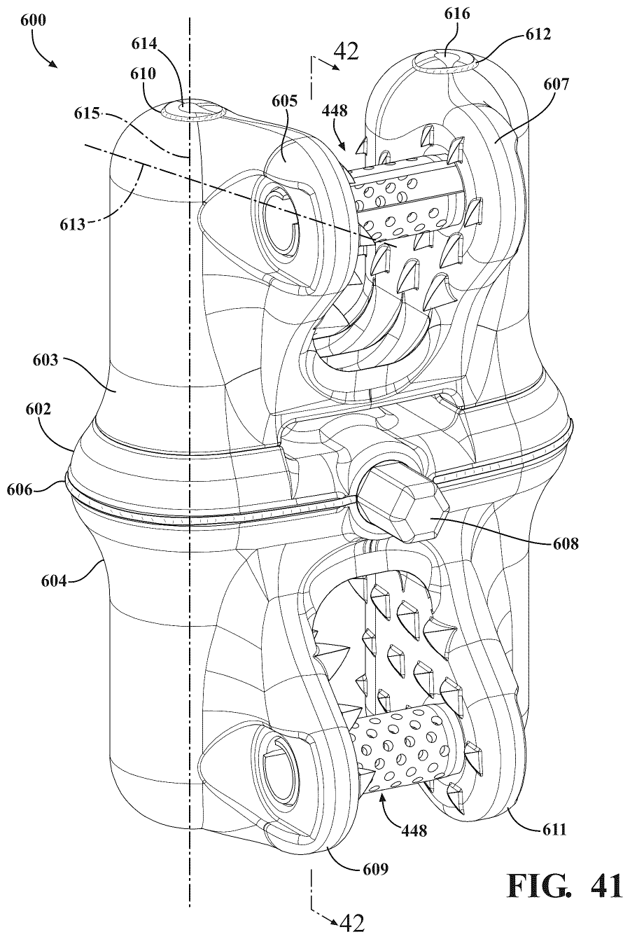

Proceeding to FIG. 41, a perspective view is generally shown at 600 of a further variant of a spinal worm screw jack having upper split body portions 602/603 and lower 604 body portion. Joining welds 606 are shown which secure the lower body portion 604 to the lower of the upper split body portions 602, with the upper most housing portion 603 being upwardly displaceable relative to the joined body portions 602/604.

Again further included are the upper and lower pocket defining bone gripping surfaces adapted for engaging the vertebral processes and such as previously described. Projecting hex bit 608 is shown and which corresponds to that previously depicted and which is formed integrally with an axial end of the central worm gear (not visible in this view however as shown previously with reference to FIG. 7 with central worm 18 with screw threads 24 for engaging the outer gears 20/22). Also shown are additional welds (see at 610/612 configured around the top edges of associated lift screws (these in addition to the laser welds previously depicted at 606 extending around the perimeter of the lower bodies).

As previously shown in related embodiments, the bone gripping surfaces each further including a “U” shaped pocket, with aligning apertures formed through the redesigned and spaced apart pairs of extending tabs (see upper tabs 605/607 and lower tabs 609/611) defining each of the upper and lower bodies. As shown, the pairs of tabs extend in a crosswise axis (see for examples as shown at 613 for upper tabs 605/607), and as opposed to linearly relative to a length directing axis (further at 615) extending through the upper and lower bodies.

The configuration of the upper and lower body portions permits reversibility of installation in either of one-hundred and eighty degree rotated positions for securing to the first and second vertebral processes (as previously shown at 6/8 in FIG. 8). The net effect of the reversibility feature is to prevent the possibility of any mistakes by the doctor/surgical team in implanting the spinal jack in an incorrect orientation to the spinous processes (again at 6 and 8 in FIG. 8).

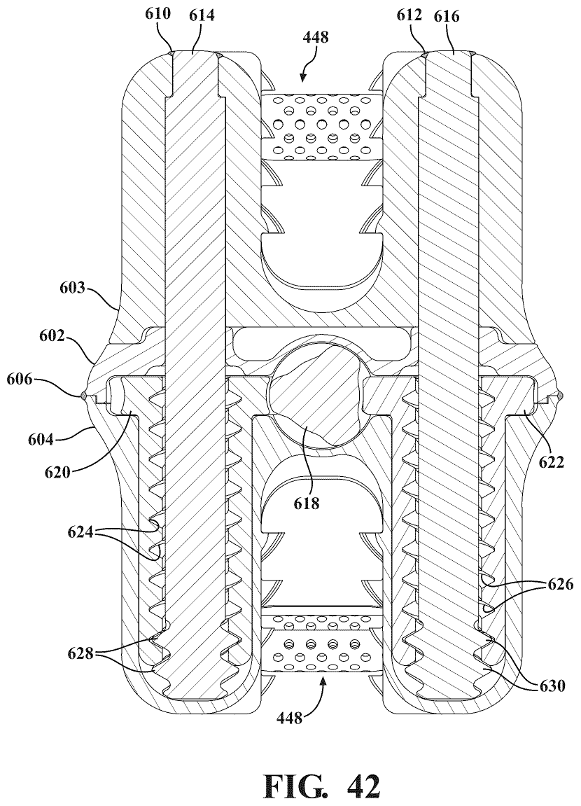

FIG. 42 is a cutaway view taken along line 42-42 of FIG. 41 and showing the spinal implant jack in a closed position with a further variant of worm gears (including central worm 618 and extended length outer worm gears 620/622 having interior threads (at 624/626) extending the length of the worm gear interiors and seating within a cavity defining interior of the lower spinal jack body. The lift screws 614 and 616 each further include a minimal number of exterior threads (628/630) at their bottom ends which are threadably engaged to the bottom interior threaded locations of the outer worm gears 620/622 in the closed position, with the lift screws seating within mating recessed profiles defined in the interior of the upper spinal body 603. Also shown are the arrangement of the upper and lower rows of gripping teeth exhibited on opposing inward surfaces of each of the upper and lower “U” shaped pockets, with the rows of gripping teeth presented in increasing dimension are shown along a direction of implantation, and as previously depicted in FIG. 40 at 508/510/512/514.

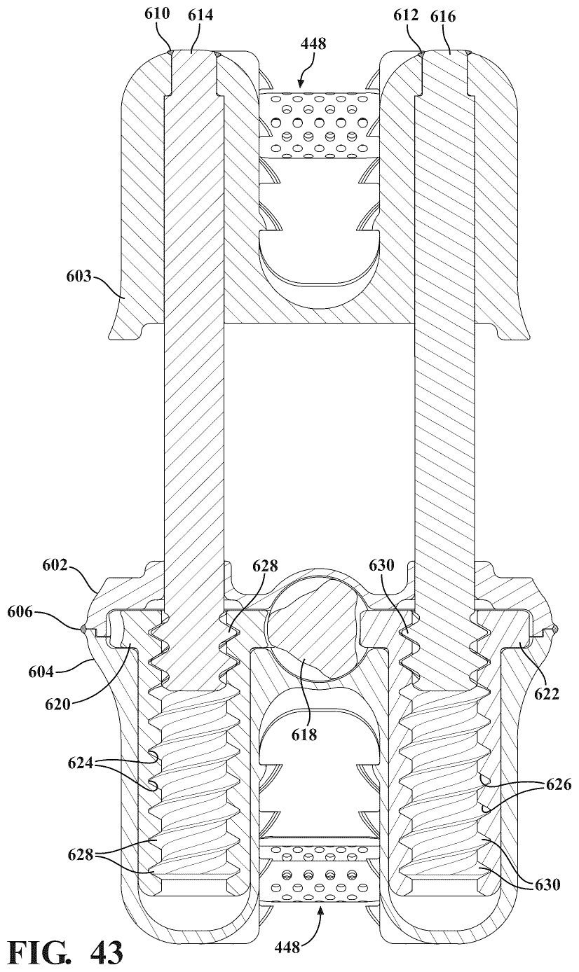

Succeeding FIG. 43 presents an expanded position of the spinal implant of FIG. 42 and showing the upper most spinal body portion 603 elevated above the welded lower portions 602/604, via the upwardly influencing displacement of the lift screws 614/616, this again in order to properly orient the spinous processes 6/8 via the actuation of the worm gear mechanism.

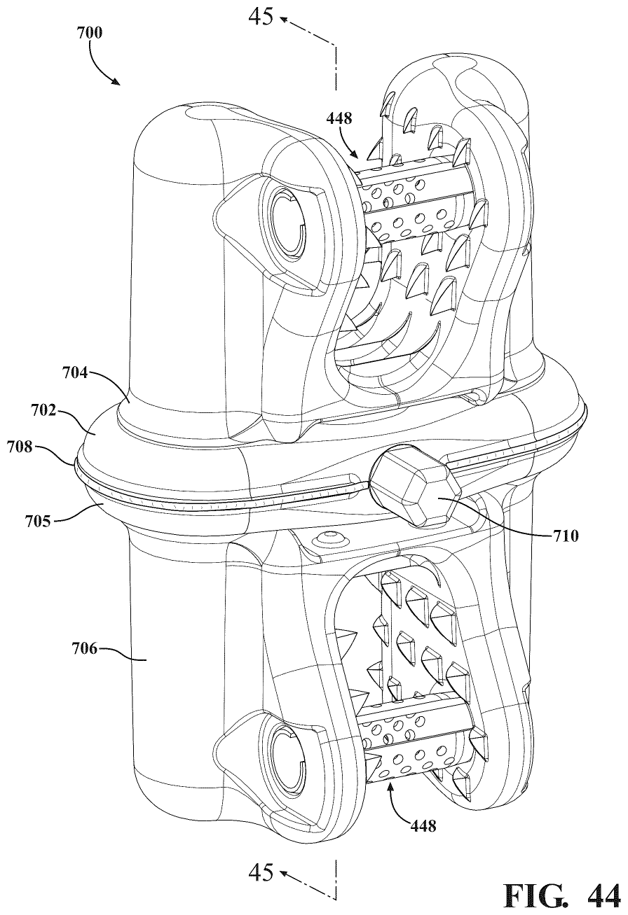

Proceeding to FIG. 44, a perspective view is shown at 700 of a further variant of a spinal worm screw jack, similar in respects to that previously shown in FIG. 41, and again having upper split body portions 702/704 and lower split body portions 705/706. Joining welds 708 are shown which secure the lowermost of the upper body portion 702 to the uppermost 705 of the lower split body portions, with both the upper most housing portion 704 and lowermost housing portion 706 being simultaneously outwardly displaceable relative to the intermediate joined body portions 702/705 as will be further described.

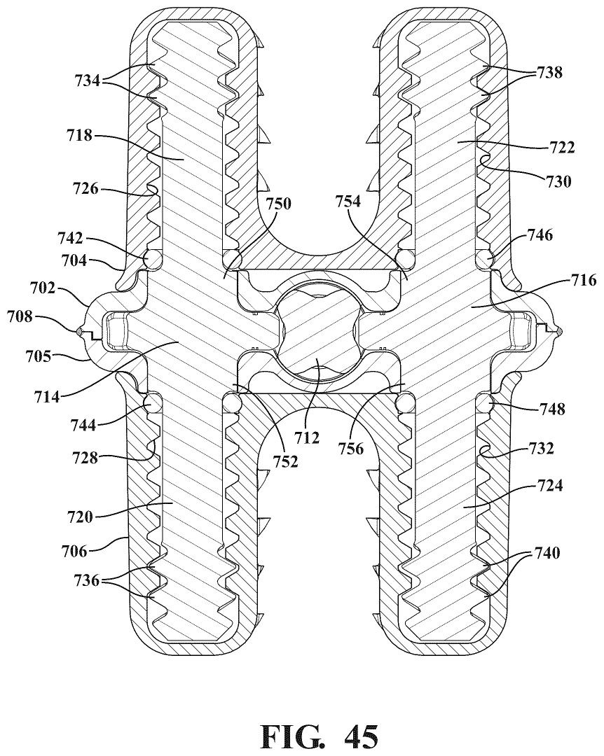

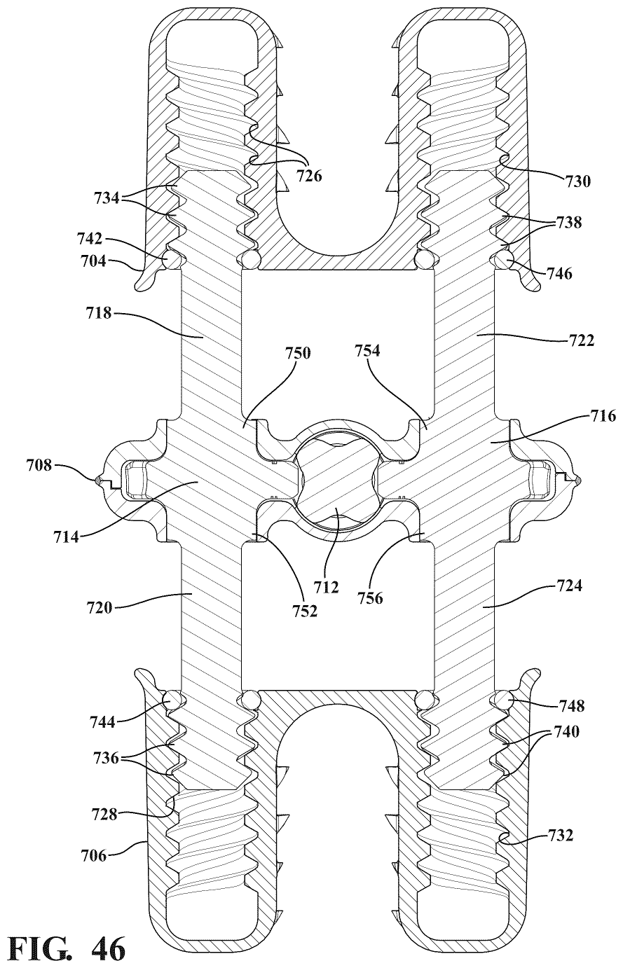

A projecting hex bit 710 is shown which corresponds to that previously depicted and which is formed integrally with an axial end of the central worm gear (not visible in this view with further reference to FIGS. 45-46, however again as shown previously with reference to FIG. 7 with central worm 18 with screw threads 24 for engaging the outer gears 20/22).

As with the prior embodiment of FIG. 41, the upper and lower body portions further including bone gripping surfaces adapted for engaging the vertebral processes, the bone gripping surfaces each further including a “U” shaped pocket with aligning apertures formed through the spaced extending tabs defining each of the “U” shaped pockets (these further arranged in a crosswise forward projecting manner relative to an extending axial length of the overall spinal implant body as shown in FIG. 41).

Similar to the embodiment of FIG. 41, the spinal jack 700 is reversible, with the upper and lower body portions having a complementing shape and configuration which permits installation in either of one-hundred and eighty degree rotated positions for securing the spinal jack to the first and second vertebral processes.

FIG. 45 presents a cutaway view taken along line 45-45 of FIG. 44 and showing the spinal implant jack in a closed position with a further variant of worm gears and lift screws for elevating the upper spinal body portion relative to the lower spinal body portion. The worm gear configuration includes a central worm 712 which is integrated into the hex bit 710. Unlike earlier variants which include both outer worm gears and thread-ably rotationally engaging lift screws, the present variant substitutes combination outer gears and lift screws as integrated components, each including central outer gear portions 714 and 716 in beveled arrangement with the central worm gear 712.

Upper and lower integrally extending lift screw stems are depicted respectively at 718/720 for outer gear portion 714 and at 722/724 for outer gear portion 716. The axially extending interiors of both the upper most housing portion 704 and lower most housing portion 706 include aligning interiorly threaded profiles, including at 726/728 and 730/732 for receiving the integrated worm gears (714/716) and opposite and integral projecting lift screw stems (718/720 and 722/724).

As shown, a minimal number of exterior threads are depicted on each of the lift screw stems (see at 734/736 for stems 718/720 and further at 738/740 for stems 722/724). The cavity defining axial extending interiors of both the upper 704 and lower 706 housing portions are closed with the outer worm gear portions 714/716 and corresponding upper/lower integrated stems 718/720 and 722/724 designed for threadably and axially displacing relative to each other upon rotation of the central worm gear for simultaneously displacing the upper and lower spinal bodies.

As further shown, pairs of upper and lower plastic or silicone O-rings (at 742/744 and 746/748) are rotatably supported at upper and lower opposing angled interface edges (further at 750/752 and 754/756) defining the annular boundaries between the central outer worm gear portions 714/716 and the upper and lower pairs of exteriorly threaded stem portions and operate to seral body fluids out of the threads, as well as providing a stopping feature for the interfacing screws.

FIG. 46 presents an expanded position of the spinal implant of FIG. 45 and depicting the upper and lower spinal bodies simultaneously displaced relative to each other upon the user rotating the hex bit 710 integrating the internal worm 712. It is further worth noting that the relative height adjustment of this variant is double that of the previous embodiments in which the upper spinal body is displaced only relative to the static positioned lower spinal body, again owing to the particular design configuration between the outer worm gears and separate threadably and rotatably engaged lift screws).

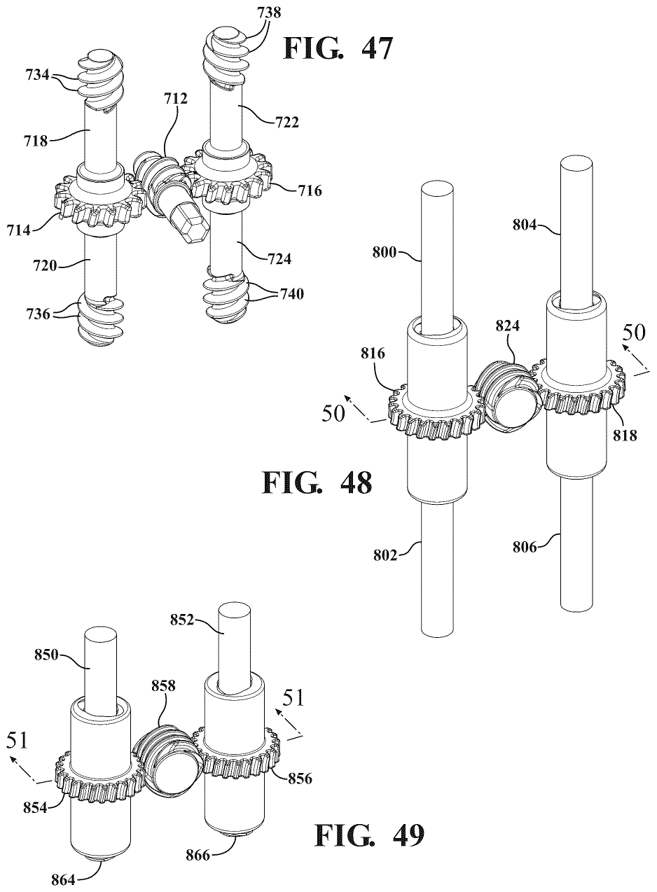

Proceeding to FIG. 47, presented is a sectional view of a non-limiting example of a worm gear arrangement according to the present invention, such as previously depicted in the spinal jack of FIGS. 45-46 and including integrated outer worm gears and integrated stem portions for simultaneously displacing the uppermost and lowermost spinal body portions or housings.

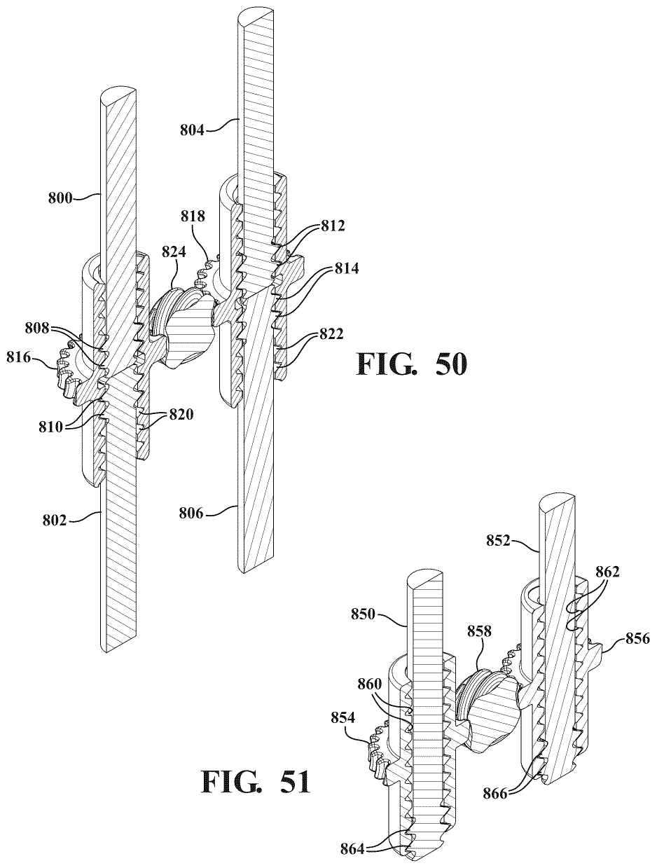

FIG. 48 presents a sectional view of a further non-limiting example of a worm gear arrangement in which individual pairs of upper/lower split stems (at 800/802 and 804/806) including smooth/non-threaded outer ends and exteriorly threaded inner opposing ends (see at 808/810 and 812/814 in cutaway of FIG. 50), these contained within outer rotatable gears 816 and 818 having interior threads 820 and 822 extending along an annular or sleeve shaped interior for simultaneously displacing the opposing pairs of stems and, by extension, the upper and lower spinal body portions (not shown) which can include inner receiving cavities which receive the stems.

FIG. 50 presents a cutaway taken along line 50-50 of FIG. 48 and illustrating the internal mating rotary threaded arrangement established between the opposing inner ends of each of the individual pairs of upper/lower split stems 800/802 and 804/806 contained within the outer rotatable gears 816 and 818, shown in the closed position and again for simultaneously displacing the upper and lower spinal body portions upon rotation of the central worm gear, at 824.

FIG. 49 presents a sectional view of a yet further non-limiting example of a worm gear arrangement in which further modified and individual split stems 850 and 852 are contained within outer rotatable gears 854 and 856 for displacing the upper spinal body portion (again not shown) relative to the lower spinal body portion (not shown).

FIG. 51 presents a cutaway view taken along line 51-51 of FIG. 49 of the further non-limiting example of worm gear arrangement and illustrating the internal mating rotary threaded arrangement established between the individual split stems 850/852 contained within the outer rotatable gears, at 854/856, for displacing the upper spinal body portion relative to the lower spinal body portion upon rotation of the central worm gear 858.

As best shown in the cutaway of FIG. 51, the outer rotatable gears 854/856 are similarly constructed as shown at 816/818 in FIG. 50 and include interior axial extending threads 860/862 which threadably receive bottom most exterior threads 864/866 configured upon the bottom ends of the stems 850/852. Without limitation, the variant of FIG. 51 can be incorporated into a spinal implant of smaller/shorter overall configuration (as compared to the variant of FIG. 50) and which can operate with a reduced range of overall height adjust-ability.

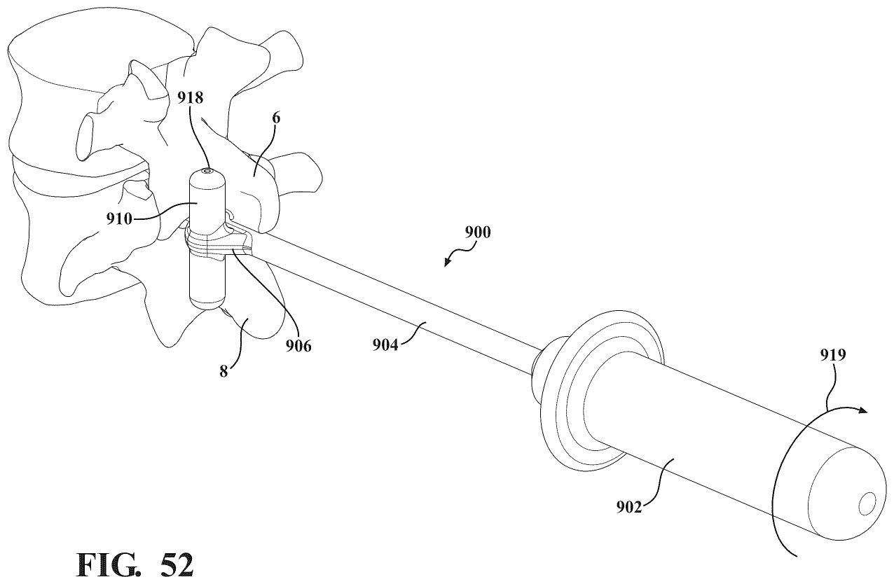

Proceeding to FIG. 52, presented is an environmental illustration of a bone preparation tool, generally at 900, this forming a part of a tool kit assembly including individual tools for providing each of preparation, implantation and fixation for installing a spinal implant jack according to the present invention. The preparation tool 900 provides for grinding or shaving of opposite facing surfaces for each of the spinous processes 6 and 8, which subsequently receive the “U” shaped pockets and opposing gripping portions associated with the upper and lower spinal body portions. In a preferred embodiment, the preparation tool simultaneously grinds the opposite surfaces of the pair of successive spinous processes in order to establish the desired installation area (or landing surfaces) prior to installation of the spinal jack as will be further described.

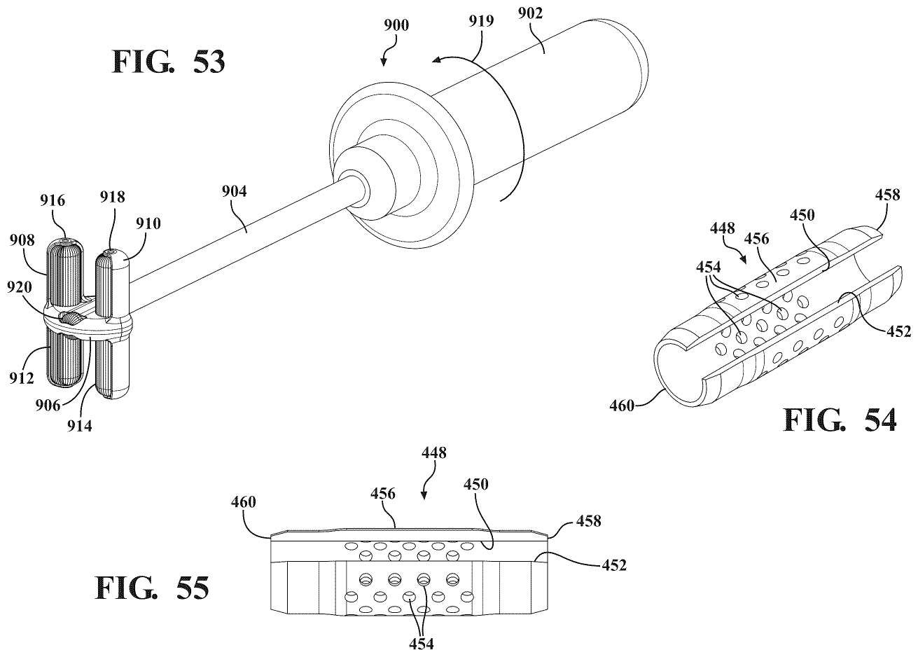

As is further shown in the rotated view of FIG. 53, the preparation tool includes a handle 902 with extending stem 904 which terminates at a forward end portion 906, in turn supporting a pair of crosswise extending hubs 908/910 which, as shown, extend both above and below the integrally supporting forward end portion 906. A pair of vertically oriented and rotary driven blades 912/914 are rotatably supported within the hubs 908/910, such as along axial pinned locations 916/918. The blades 912/914 can, without limitation, include multiple individual shaving edges which are arrayed in a circumferential arrangement.

The preparation tool can also include a portable and battery powered electric motor incorporated into the handle and which, when actuated (such as by a switch or rotating an outer gripping portion the handle) rotates an interior shaft (not shown) extending through a hollow interior of the stem 904. Without limitation, the preparation tool is operated by holding the shaft 904 and rotating the handle 902 in order to operate the blades 912/914, the speed of which can be variable depending on the degree of rotation of the handle (see at 919).

A forward supported end of the interior shaft is shown at 920 in FIG. 53 supported to the forward end portion 906, such that an internal bevel gearing arrangement can provide for converting rotation of the inner shaft to the elongated and rotary blades 912/914 for grinding or shaving/conditioning the spinous process surfaces of the affected vertebrae processes for subsequently seating the respective upper and lower spinal body halves. A vacuum tube (not shown) can be attached to the tube in order to evacuate the grinding bone debris during the initial conditioning the spinous process surfaces.

As further shown, the configuration of the rotary blades 912/914 being utilized enables the symmetrical shaving of both pairs of opposite landing surfaces associated with both the upper and lower vertebrae for receiving each of the reversible spinal jacks not limited to those as previously depicted in FIGS. 41 and 44. Alternatively, the preparation tool can be modified to customize the shaping/conditioning the spinous process surfaces in order to mount any alternate design variant of spinal jack which may not be reversibly mountable.

FIG. 56 presents an initial implantation step of a spinal jack by an implantation tool, generally at 950, which is similar in numerous respects to that previously shown at 296 in FIGS. 18-21, and which is illustrated environmentally and which provides for locating and resistive seating of the spinal implant jack according to any of the disclosed embodiments herein, not limited to the reversible jack implants 600 and 700 previously described, against the spinous processes 6/8 of the successive vertebrae.

Similar to the original variant 296, the insertion or implantation tool includes an elongated neck or shank 952 (see as compared to at 300 in FIG. 18), which extends from a rear “T” shaped handle 954 including annular flattened surface 956 to a forward most rectangular shaped end location or portion 958 (see also previously shown at 298) which, as previously described, communicates a rear induced rotation provided by an attachable adjustment component 960 (similar to the tool bit driver 304 in FIG. 20) and having a forward socket portion 962 which seats within a central open annular interior 964 surrounded by the annular flattened surface 956.

In a first implantation step, the spinal jack (by non-limiting example again shown at 600) is fixedly secured to upper and lower shaft tabs associated with the forward end portion 958 of the insertion tool and such as which can be arranged above and below a central recessed hex bit portion (not shown) which receives the projecting central worm gear hex bit associated with the spinal jack. Anchoring of the spinal jack to the insertion tool prior to implantation can be accomplished by partially separating the opposing jack halves in order to achieve a minimal separation distance adequate for inserting the tabs of the forward rectangular end portion 958, following which the hex nut is reverse rotated by hex bit in order to employ the worm gear to firmly reverse/inwardly displace the jack halves and to clamp inwardly against the opposing support tabs of the forward rectangular support 958.

With the spinal jack fixedly secured to the insertion tool, the surgeon inserts and manipulates the implant by twisting and turning the tool in order to affix and clamp down the upper and lower “U” shaped receiving pockets with gripping portions around and against the previously ground/shaved and reconditioned landing surfaces of the spinous processes. To complete the placement, the surgeon can pound against the exposed flattened surface 956 (such as with a mallet) in order to set the implant as close as possible to the base of the spinous processes.

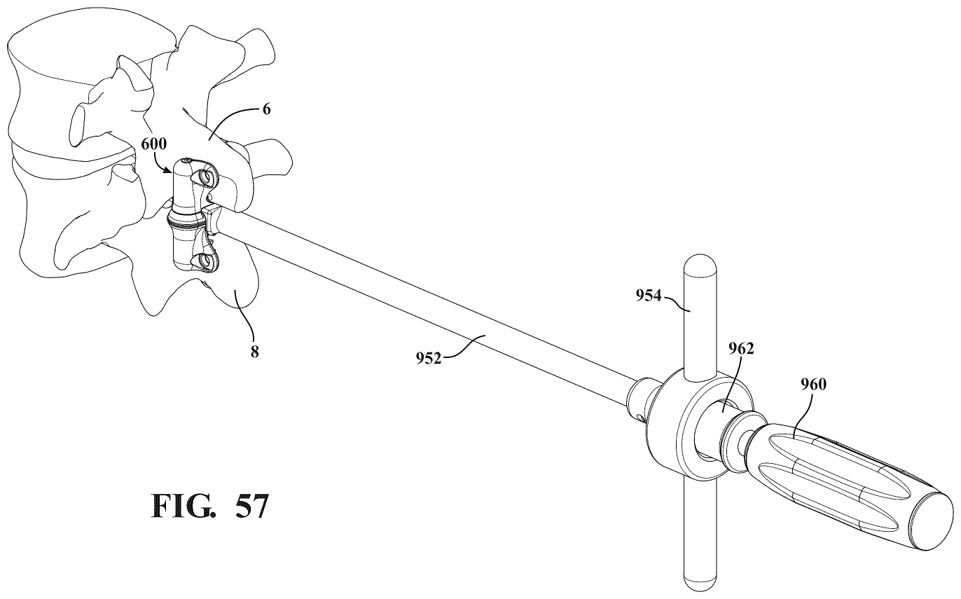

FIG. 57 is succeeding view of FIG. 56 and depicting the handle shaped adjustment component 960 with forward socket 962 reattached to the central open annular interior 964 surrounded by the annular flattened surface 956 of the implantation tool of FIG. 56. As previously described in the insertion tool variant of FIGS. 18-21, the handle is subsequently rotated in order to expand the jack halves to achieve a proper orientation of the vertebrae via the mounted spinous processes.

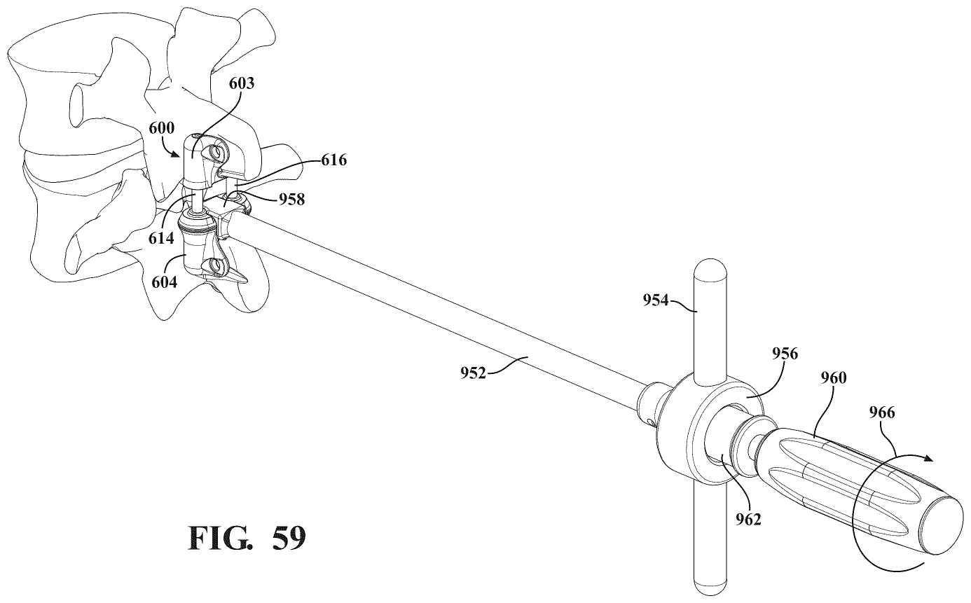

The step of expanding the jack halves (with succeeding reference to FIG. 59) can occur either prior or subsequent to the installation of the split rivets 448, as is now depicted in reference to the FIG. 58 which further depicts the fixation tool, generally at 1000, employed for driving the previously described split rivets 448 between the upper and lower ears or lobes of the spinal body halves and through the intervening bone of the spinous processes 6/8, again without the need for pre-drilling apertures through the bone. As further shown in FIG. 58, the fixation tool 1000 is configured in non-limiting arrangement as an in-line plier device having compressible end handles 1002/1004 with forward extending linkage arms 1006/1008 which are rotatably coupled by a rear pin/shaft arrangement at 1010.

Forward ends of the linkage arms 1006/1008 include additional end pin and shaft locations (at 1012 and 1014), to which are respectively secured forward most rivet installation members 1016 and 1018, these further seating therebetween a force multiplying overlapping fulcrum arrangement shown by overlapping and pivotally secured members 1020 and 1022. The members 1020/1022 are pinned at their ends to the pin and shaft support locations 1012/1014 and, forward of the overlapping interface between the members, each terminating in a pin 1024 and 1026 respectively seating within a crosswise extending channel 1028/1030.

As further shown, the split rivet or roll pin 448 is located within a received cavity associated with a branching forward end 1032 of the installation member 1018 which is located against an exterior surface of a first selected mounting tab or lobe on one side of the upper or lower implant body. A further branching forward support or anvil portion 1034 is further depicted aligning with an opposite exterior surface of an aligning mounting tab or lobe located on the other side of either of the upper or lower implant bodies.

A forward most extending portion 1036 of the rivet installation member 1016 seats within a three dimensionally configured interior pocket defined by the branching forward end 1032 of the other linkage actuated installation member 1018. Upon squeezing the rear handles 1002/1004 together, a force multiplier is exerted throughout the linkage, including the travel of the pins 1024/1026 within the associated channels or slots 1028/1030 in order to achieve a controlled inward displacement of the forward extending portion 1036 actuating inwardly against the rivet 448, in order to drive the forward annular blade edge of the rivet as previously described through the spinous process bone so that it aligns and seats through the opposing aligning aperture of opposite located mounting tab, ear or lobe as previously described. Without limitation, the fixation tool can be reconfigured or redesigned as required in order to mount any style of rivet (not limited to that disclosed herein).

FIG. 59 depicts a final installation step again using the implantation tool 950, and following either of the insertion step of FIG. 57 or rivet fixation step of FIG. 58, and by which the reattached handle component 960 is rotated (see arrow 966) in order to expand the upper and lower spinal bodies (again depicted in non-limiting example by upper body 603 and lower body 604 of the exemplary reversible spinal jack 600) into a desired expanded position for properly aligning the vertebrae. Expansion of the jack halves by the rotation of the handle 960 and socket 962 (such again occurring via an internal rotary shaft which drives the forward hex bit) causes the jack halves to unseat and release from the forward most located support tabs of the insertion tool, with the spinal jack halves being expanded to the extent necessary to establish the desired fixed separation distance for supporting the spinous processes of the succeeding vertebrae in their desired arrangement.

Having described my invention, other and additional preferred embodiments will become apparent to those skilled in the art to which it pertains, and without deviating from the scope of the appended claims. The detailed description and drawings are further understood to be supportive of the disclosure, the scope of which being defined by the claims. While some of the best modes and other embodiments for carrying out the claimed teachings have been described in detail, various alternative designs and embodiments exist for practicing the disclosure defined in the appended claims.

The foregoing disclosure is further understood as not intended to limit the present disclosure to the precise forms or particular fields of use disclosed. As such, it is contemplated that various alternate embodiments and/or modifications to the present disclosure, whether explicitly described or implied herein, are possible in light of the disclosure. Having thus described embodiments of the present disclosure, a person of ordinary skill in the art will recognize that changes may be made in form and detail without departing from the scope of the present disclosure. Thus, the present disclosure is limited only by the claims.

In the foregoing specification, the disclosure has been described with reference to specific embodiments. However, as one skilled in the art will appreciate, various embodiments disclosed herein can be modified or otherwise implemented in various other ways without departing from the spirit and scope of the disclosure. Accordingly, this description is to be considered as illustrative and is for the purpose of teaching those skilled in the art the manner of making and using various embodiments of the disclosure. It is to be understood that the forms of disclosure herein shown and described are to be taken as representative embodiments. Equivalent elements, materials, processes or steps may be substituted for those representatively illustrated and described herein. Moreover, certain features of the disclosure may be utilized independently of the use of other features, all as would be apparent to one skilled in the art after having the benefit of this description of the disclosure. Expressions such as “including”, “comprising”, “incorporating”, “consisting of”, “have”, “is” used to describe and claim the present disclosure are intended to be construed in a non-exclusive manner, namely allowing for items, components or elements not explicitly described also to be present. Reference to the singular is also to be construed to relate to the plural.

Further, various embodiments disclosed herein are to be taken in the illustrative and explanatory sense, and should in no way be construed as limiting of the present disclosure. All joinder references (e.g., attached, affixed, coupled, connected, and the like) are only used to aid the reader's understanding of the present disclosure, and may not create limitations, particularly as to the position, orientation, or use of the systems and/or methods disclosed herein. Therefore, joinder references, if any, are to be construed broadly. Moreover, such joinder references do not necessarily infer that two elements are directly connected to each other.

Additionally, all numerical terms, such as, but not limited to, “first”, “second”, “third”, “primary”, “secondary”, “main” or any other ordinary and/or numerical terms, should also be taken only as identifiers, to assist the reader's understanding of the various elements, embodiments, variations and/or modifications of the present disclosure, and may not create any limitations, particularly as to the order, or preference, of any element, embodiment, variation and/or modification relative to, or over, another element, embodiment, variation and/or modification.

It will also be appreciated that one or more of the elements depicted in the drawings/figures can also be implemented in a more separated or integrated manner, or even removed or rendered as inoperable in certain cases, as is useful in accordance with a particular application. Additionally, any signal hatches in the drawings/figures should be considered only as exemplary, and not limiting, unless otherwise specifically specified.

1. A spinal jack adapted for installation between first and second vertebral processes, comprising:
an upper body portion and a lower body portion;
each of said upper and lower body portions further including bone gripping surfaces adapted for engaging the vertebral processes, said bone gripping surfaces each further including a “U” shaped pocket, aligning apertures formed through spaced extending tabs defining each of said “U” shaped pockets;
rivets inserted between the aligning apertures and adapted to being pressed through a bone of the vertebral processes for securing said upper and lower body portions to the vertebral processes, said rivets further having a split tubular shape along its extending length and including apertures distributed across a width and circumference thereof for facilitating bone in-growth; and
a worm gear mechanism for adjusting a separation distance between said vertebral processes via said upper and lower body portions and including a rotatable worm incorporated into said body for engaging a pair of outer gears communicating said worm with a pair of stems extending between communicating interiors of said upper and lower body portions for displacing said upper body portion relative to said lower body portion.

2. The spinal jack of claim 1, said rivets further including a larger central diameter in comparison to first and second end located diameters for affixing in position within the vertebral bone.

3. The spinal jack of claim 1, further comprising a bit engaging portion integrally formed with said worm and projecting from a surface of said lower body portion.

4. The spinal jack of claim 1, further comprising said “U” shaped pocket exhibiting gripping teeth and facilitating unidirectional installation against the processes along with preventing detachment from uneven surfaces of the processes once installed.

5. The spinal jack of claim 4, further comprising said gripping teeth comprising individual rows being provided with incrementing height, with the shortest being an initial row engaging the vertebral bone, each succeeding row of teeth being progressively larger for engaging new bone during installation.

6. The spinal jack of claim 1, said upper and lower body portions further comprising a medical grade titanium.

7. The spinal jack of claim 1, said lower body portion incorporating a recessed cavity for receiving said rotatable worm and said outer gears.

8. The spinal jack of claim 7, further comprising additional cavities configured into said body portions for seating said displaceable stems.

9. The spinal jack of claim 1, said lower body portion further comprising first and second subset portions incorporating said worm gear mechanism.

10. The spinal jack of claim 1, further a latticing patterns formed in the bone gripping surfaces.

11. A spinal jack adapted for installation between first and second vertebral processes, comprising:
an upper body portion and a lower body portion;
each of said upper and lower body portions further including bone gripping surfaces adapted for engaging the vertebral processes, said bone gripping surfaces each further including a “U” shaped pocket, aligning apertures formed through spaced extending tabs defining each of said “U” shaped pockets;
each of said “U” shaped pockets of said upper and lower body portions having a shape and configuration which permits reversible installation in either of one-hundred and eighty degree rotated positions for securing to the vertebral processes;
rivets inserted between the aligning apertures and adapted to being pressed through a bone of the vertebral processes for securing said upper and lower body portions to the vertebral processes; and
a worm gear mechanism for adjusting a separation distance between said vertebral processes via said upper and lower body portions and including a rotatable worm incorporated into said body for engaging a pair of outer worm gears communicating said worm with a pair of stems extending between communicating interiors of said upper and lower body portions for displacing said upper body portion relative to said lower body portion.

12. The spinal jack of claim 11, said rivets further comprising a split tubular shape along its extending length and including apertures distributed across a width and circumference thereof for facilitating bone in-growth.

13. The spinal jack of claim 11, said rivets further including a larger central diameter in comparison to first and second end located diameters for affixing in position within the vertebral bone.

14. The spinal jack of claim 11, further comprising a bit engaging portion integrally formed with said worm and projecting from a surface of said lower body portion.

15. The spinal jack of claim 11, further comprising said “U” shaped pocket exhibiting gripping teeth and facilitating unidirectional installation against the processes along with preventing detachment from uneven surfaces of the processes once installed.

16. The spinal jack of claim 15, further comprising said gripping teeth comprising individual rows being provided with incrementing height, with the shortest being an initial row engaging the vertebral bone, each succeeding row of teeth being progressively larger for engaging new bone during installation.

17. The spinal jack of claim 11, said upper and lower body portions further comprising a medical grade titanium.

18. The spinal jack of claim 11, said lower body portion incorporating a recessed cavity for receiving said rotatable worm and said outer gears, additional cavities configured into said body portions for seating said displaceable stems.

19. The spinal jack of claim 11, said lower body portion further comprising first and second subset portions incorporating said worm gear mechanism.

20. The spinal jack of claim 11, further a latticing patterns formed in the bone gripping surfaces.
